(19) United States
(12) Patent Application Publication
(10) Pub. No.: US 2026/0083552 A1
(43) Pub. Date: Mar. 26, 2026

(54) COMPLIANCE IMPLANT DEVICES WITH ELASTIC TUBES

(71) Applicant: EDWARDS LIFESCIENCES CORPORATION, Irvine, CA (US)

(72) Inventors: Amit Peer, Rehovot (IL); Bezalel Haberman-Browns, Pardes Hanna (IL)

(73) Assignee: EDWARDS LIFESCIENCES CORPORATION, Irvine, CA (US)

(21) Appl. No.: 19/172,243

(22) Filed: Apr. 7, 2025

Related U.S. Application Data
(63) Continuation of application No. PCT/US2023/076318, filed on Oct. 7, 2023.
(60) Provisional application No. 63/378,920, filed on Oct. 10, 2022.

(51) Int. Cl. A61F 2/24

(52) U.S. Cl. CPC A61F 2/2418; A61F 2220/0008

(57) ABSTRACT

Devices, systems, and/or methods can provide compliance characteristics to fluid vessels. For example, an implant device can include an anchoring structure to anchor the implant device to a blood vessel, heart valve, or other anatomy. The anchoring structure can be coupled to an elastic tube that is configured to expand. The elastic tube can include a prosthetic valve to permit fluid to flow through the implant device.

### RELATED APPLICATION(S)

This application is a continuation of International Patent Application No. PCT/US23/76318, filed Oct. 7, 2023, which claims the benefit of U.S. Provisional Patent Application No. 63/378,920, filed on Oct. 10, 2022, the complete disclosures of which are hereby incorporated by reference in their entireties.

### BACKGROUND

### Field

The present disclosure generally relates to the field of medical implant devices.

### Description of Related Art

Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and/or systems that facilitate the restoration of compliance characteristics to undesirably stiff blood vessels and other anatomy. For example, an implant device can include an anchoring structure configured to anchor the implant device to a blood vessel, heart valve, or other anatomy. The anchoring structure can be coupled to an elastic tube that is configured to expand and contract to change a volume of blood or other fluid passing therethrough. Such change in volume can allow the blood vessel to mimic compliance of a healthy blood vessel and/or otherwise promote blood flow during, for example, a phase of the cardiac cycle.

For purposes of summarizing the disclosure, certain aspects, advantages, and/or features are described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIG. 1A illustrates an example representation of a heart and associated vasculature having various features relevant to one or more examples of the present disclosure.

FIG. 1B illustrates an example healthy aorta.

FIG. 1C illustrates an example unhealthy aorta.

FIG. 2A provides a side view of a compliant blood vessel radially contracting/recoiling during the diastolic phase of the cardiac cycle.

FIG. 2B provides a side view of a compliant blood vessel experiencing expansion during the systolic phase of the cardiac cycle.

FIG. 2C provides a cross-sectional view of a compliant blood vessel radially contracting/recoiling during the diastolic phase of the cardiac cycle.

FIG. 2D provides a cross-sectional view of a compliant blood vessel experiencing expansion during the systolic phase of the cardiac cycle

FIGS. 3A and 3B illustrate cross-sectional views of a blood vessel that is relatively stiff.

FIG. 4 is a graph illustrating blood pressure over time in an example healthy patient.

FIG. 5 is a graph illustrating blood pressure over time in an example patient having reduced aortic compliance.

FIGS. 6A, 6B, 6C, and 6D provide perspective, side, front, and cross-sectional views, respectively, of an example device that can be configured to enhance compliance of a fluid vessel.

FIG. 7A illustrates an example implant device with a prosthetic valve.

FIGS. 7B, 7C, and 7D illustrate example prosthetic valves that can be implemented with the implant device of FIG. 7A in accordance with one or more examples.

FIG. 8 illustrates another example prosthetic valve that can be implemented with an implant device in accordance with one or more examples.

FIG. 9 illustrates the example implant device of FIGS. 6A-6D disposed within anatomy of a patient in accordance with one or more examples.

FIGS. 10A and 10B illustrate operation of the example implant device of FIGS. 6A-6D during various phases of the cardiac cycle in accordance with one or more examples.

FIGS. 11A, 11B, and 11C provide side, front, and cross-sectional views, respectively, of another example implant device that can be configured to enhance compliance of a fluid vessel.

FIGS. 12A and 12B illustrate the example implant devices of FIGS. 11A-11C disposed within anatomy of a patient in accordance with one or more examples.

FIGS. 13A and 13B illustrate operation of the example implant device of FIGS. 11A-11C during various phases of the cardiac cycle in accordance with one or more examples.

FIG. 14 provides a side view of another example implant device that can be configured to enhance compliance of a fluid vessel.

FIG. 15 illustrates the example implant device of FIG. 14 disposed within anatomy of a patient in accordance with one or more examples.

FIGS. 16A, 16B, 16C, 16D, 16E, and 16F illustrate a flow diagram for a process for implanting an implant device in accordance with one or more examples.

FIGS. 17A, 17B, 17C, 17D, 17E, and 17F provide images of the implant device and certain anatomy corresponding to operations of the process of FIGS. 16A, 16B, 16C, 16D, 16E, and 16F according to one or more examples.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for ease of description to describe the positional relationship between element(s)/structures(s), as illustrated in the drawings.

Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

Any of the example methods and/or structures disclosed herein for treating a patient also encompass analogous methods and/or structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and/or the methods herein can comprise sterilization of the associated system, device, apparatus, etc. (e.g., with heat, radiation, ethylene oxide, hydrogen peroxide, etc.).

### Vascular Anatomy and Compliance

Certain examples are disclosed herein in the context of vascular implant devices, and in particular, compliance-enhancement implant devices implanted in the aorta. However, although certain principles disclosed herein can be particularly applicable to the anatomy of the aorta, the compliance-enhancement implant devices in accordance with the present disclosure can be implanted in, or configured for implantation in, any suitable or desirable blood vessels or other anatomy, such as the inferior vena cava, etc.

The anatomy of the heart and vascular system is described below to assist in the understanding of certain concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles can be prompted by signals generated by the electrical system of the heart.

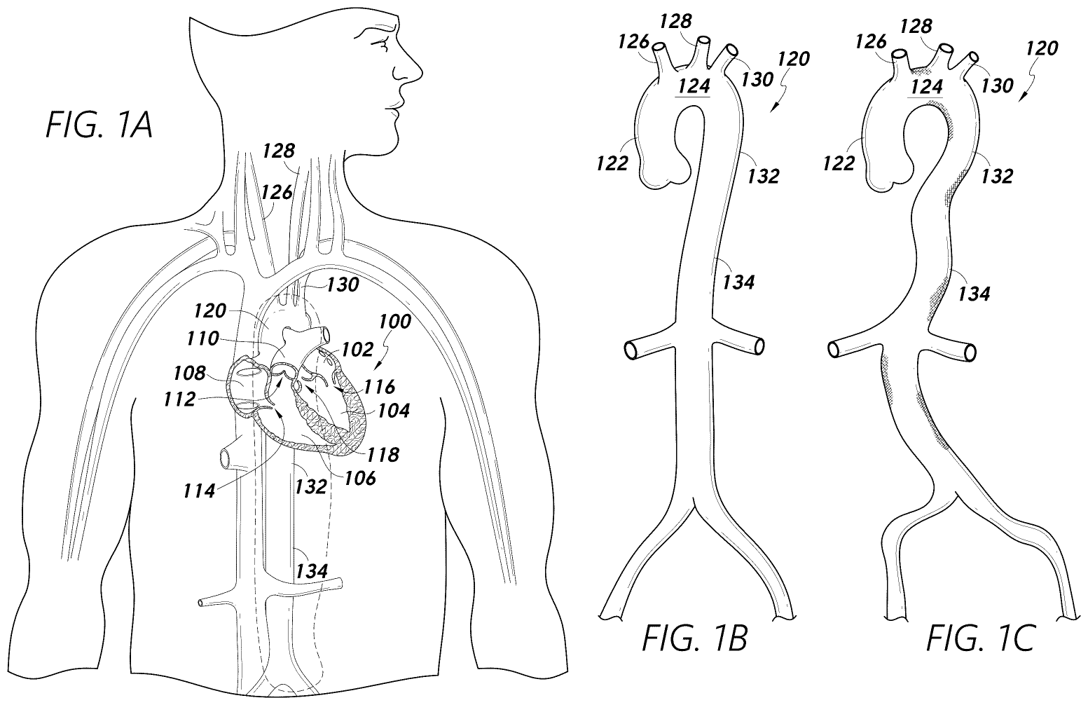

FIG. 1A illustrates an example representation of a heart 100 and associated vasculature having various features relevant to one or more examples of the present disclosure. The heart 100 includes four chambers, namely the left atrium 102, the left ventricle 104, the right ventricle 106, and the right atrium 108. In terms of blood flow, blood generally flows from the right ventricle 106 into the pulmonary artery 110 via the pulmonary valve 112, which separates the right ventricle 106 from the pulmonary artery 110 and is configured to open during systole so that blood can be pumped toward the lungs and close during diastole to prevent blood from leaking back into the heart from the pulmonary artery 110. The pulmonary artery 110 carries deoxygenated blood from the right side of the heart 100 to the lungs. The pulmonary artery 110 includes a pulmonary trunk and left and right pulmonary arteries that branch off of the pulmonary trunk, as shown.

The tricuspid valve 114 separates the right atrium 108 from the right ventricle 106. The tricuspid valve 114 generally has three cusps/leaflets and can generally close during ventricular contraction (i.e., systole) and open during ventricular expansion (i.e., diastole). The mitral valve 116 generally has two cusps/leaflets and separates the left atrium 102 from the left ventricle 104. The mitral valve 116 is configured to open during diastole so that blood in the left atrium 102 can flow into the left ventricle 104, and, when functioning properly, closes during systole to prevent blood from leaking back into the left atrium 102. The aortic valve 118 separates the left ventricle 104 from the aorta 120. The aortic valve 118 is configured to open during systole to allow blood leaving the left ventricle 104 to enter the aorta 120, and close during diastole to prevent blood from leaking back into the left ventricle 104.

The heart valves can generally comprise a relatively dense fibrous ring, referred to herein as the annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, can generally comprise finger-like projections from the ventricle wall. The valve leaflets are connected to the papillary muscles by the chordae tendineae. A wall of muscle, referred to as the septum, separates the left 102 and right 108 atria and the left 104 and right 106 ventricles.

The vasculature of the human body, which can be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior venae cavae, carry blood back to the heart.

FIGS. 1B and 1C show detailed views of example healthy and aged/stiff aortas 120, respectively. The aorta 120 is a compliant arterial blood vessel that buffers and conducts pulsatile left ventricular output and contributes the largest component of total compliance of the arterial tree. The aorta 120 includes the ascending aorta 122, which begins at the opening of the aortic valve 118 in the left ventricle 104 of the heart 100. The ascending aorta 122 and pulmonary trunk 110 twist around each other, causing the aorta 120 to start out posterior to the pulmonary trunk 110, but end by twisting to its right and anterior side. Among the various segments of the aorta 120, the ascending aorta 122 is relatively more frequently affected by aneurysms and dissections, often requiring open heart surgery to be repaired. The transition from ascending aorta 122 to aortic arch 124 is at the pericardial reflection on the aorta. At the root of the ascending aorta 122, the lumen has three small pockets between the cusps of the aortic valve 118 and the wall of the aorta 120, which are called the aortic sinuses or the sinuses of Valsalva. The left aortic sinus contains the origin of the left coronary artery and the right aortic sinus likewise gives rise to the right coronary artery. Together, these two arteries supply the heart with blood.

As mentioned above, the aorta 120 is coupled to the heart 100 via the aortic valve 118, which leads into the ascending aorta 122 and gives rise to the innominate artery 126, the left common carotid artery 128, and the left subclavian artery 130 along the aortic arch 124 before continuing as the descending thoracic aorta 132 and further the abdominal aorta 134. References herein to the aorta can be understood to refer to the ascending aorta 122 (also referred to as the “ascending thoracic aorta”), aortic arch 124, descending or thoracic aorta 132 (also referred to as the “descending thoracic aorta”), abdominal aorta 134, or other arterial blood vessel or portion thereof.

Arteries, such as the aorta 120, can utilize blood vessel compliance (e.g., arterial compliance) to store and release energy through the stretching of blood vessel walls. The term “compliance” can be used herein according to its broad and ordinary meaning, and can refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood can flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta, as shown in FIG. 1B, runs along a generally straight path, whereas an aged and/or stiffened aorta, as shown in FIG. 1C, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

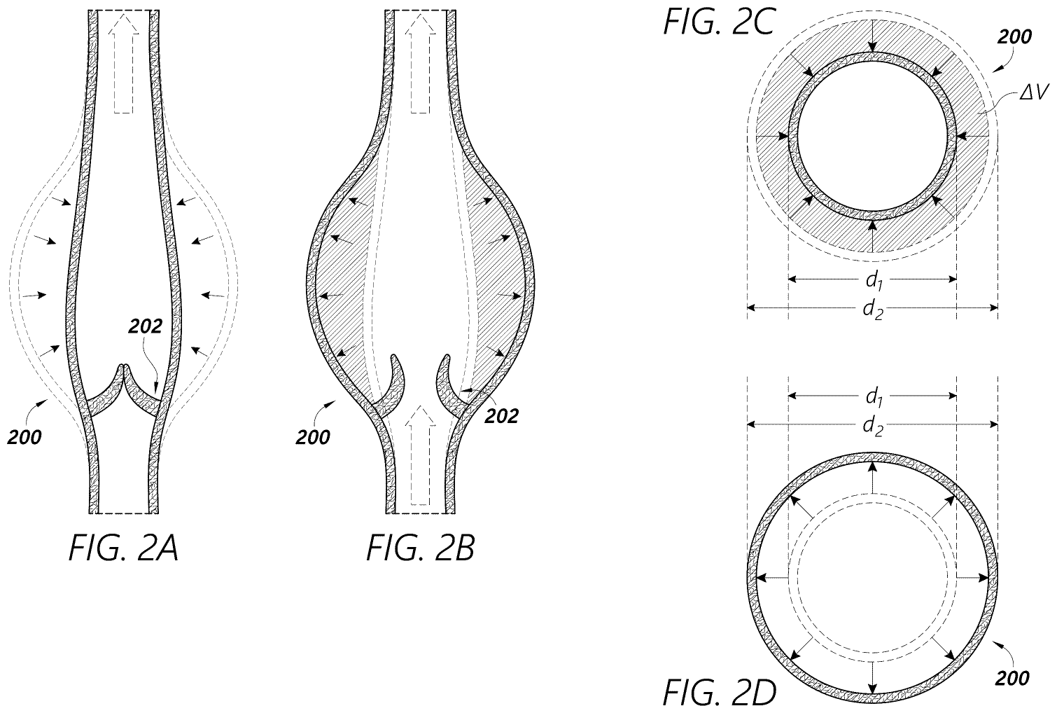

FIGS. 2A and 2C provide side and cross-sectional views, respectively, of a compliant blood vessel 200, such as an artery (e.g., aorta), radially contracting/recoiling during the diastolic phase of the cardiac cycle. FIGS. 2B and 2D provide side and cross-sectional views, respectively, of the compliant blood vessel 200 experiencing expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIG. 2C, with proper arterial compliance, a change in volume ΔV will generally occur in an artery between high-and low-pressure phases of the cardiac cycle. With respect to the aorta 200, as shown in FIGS. 2B and 2D, as blood is pumped into the aorta 200 through the aortic valve 202, the pressure in the aorta 200 increases and the diameter of at least a portion of the aorta 200 expands. A first portion of the blood entering the aorta 200 during systole may pass through the aorta 200 during the systolic phase, while a second portion (e.g., approximately half of the total blood volume) may be stored in the expanded volume ΔV caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance can have a significant effect on perfusion and/or blood pressure in some patients. For example, arteries with relatively higher compliance can be conditioned to more easily deform than lower-compliance arteries under the same pressure conditions. Compliance (C) can be calculated using the following equation, where ΔV is the change in volume (e.g., in mL) of the blood vessel, and ΔP is the pulse pressure from systole to diastole (e.g., in mmHg):

C=ΔV/ΔP (1)

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the ascending aorta. However, such devices, methods and/or concepts can be applicable in connection with any other artery or blood vessel.

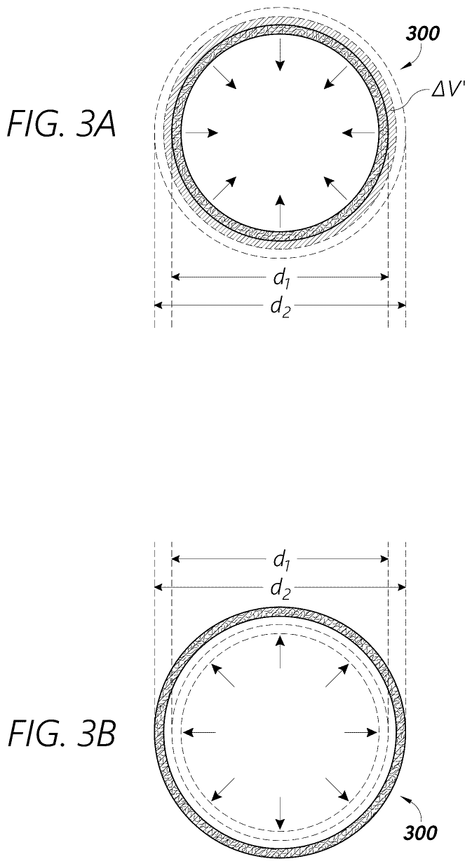

FIGS. 3A and 3B show a cross-sectional profile of a blood vessel 300 that is relatively stiff, such as the blood vessel shown in FIG. 1C, wherein the compliance of the vessel portion 300 is diminished relative to the healthy aorta as shown in FIG. 1B.

Due to the stiffness of the blood vessel wall, the blood vessel 300 can expand a relatively limited amount between diastole (shown in FIG. 3A) and systole (shown in FIG. 3B). That is, during systole, the increased fluid pressure within the blood vessel 300 can result in a relatively small and/or negligible expansion of the diameter of the blood vessel 300, as shown with respect to the difference between the contracted diameter d₁ and the expanded diameter d₂. Due to the limited expansion of the blood vessel 300, the change in volume ΔV′ in the blood vessel between phases of the cardiac cycle can likewise be limited, and therefore relatively little energy is stored in the blood vessel wall and returned to the blood circulation during low-pressure conditions, resulting in more pulsatile blood flow compared to healthy, compliant aortic tissue.

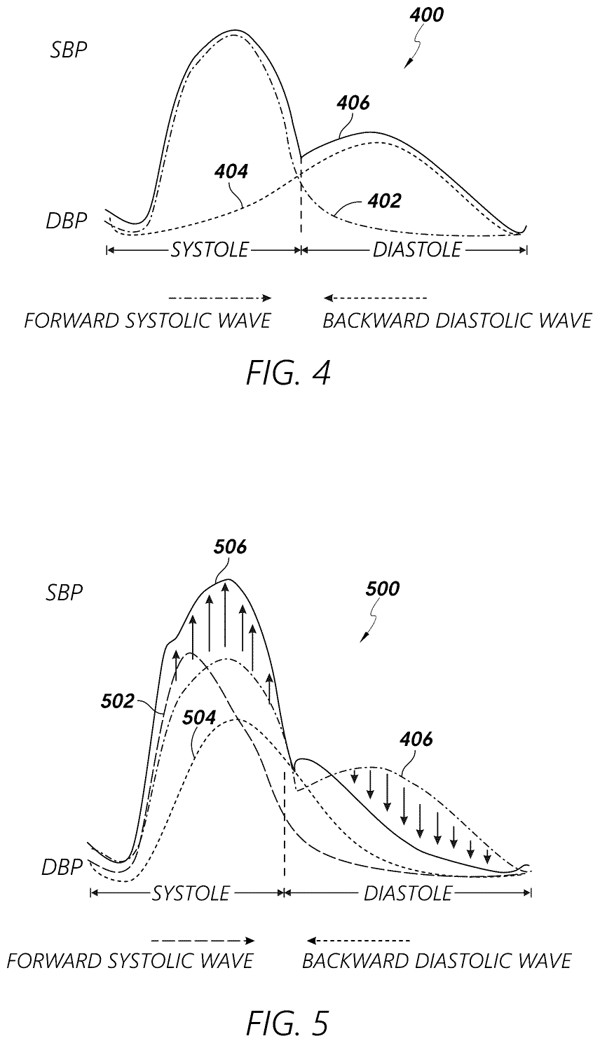

FIG. 4 is a graph 400 illustrating blood pressure over time in an example patient with a healthy, compliant aorta, wherein arterial blood pressure is represented as a combination of a forward systolic pressure wave 402 and a backward diastolic pressure wave 404. The combination of the systolic wave 402 and the diastolic wave 404 are represented by the waveform 406.

FIG. 5 is a graph 500 illustrating blood pressure over time in an example patient having reduced aortic compliance. The graph 500 shows, for reference purposes, the example combined wave 406 shown in FIG. 4. When low compliance is exhibited, less energy can be stored in the aorta compared to a healthy patient. Therefore, the systolic waveform 502 can demonstrate increased pressure during the systolic phase relative to a patient having normal compliance, while the diastolic waveform 504 can demonstrate reduced pressure during the diastolic phase relative to a patient having normal compliance. Therefore, the resulting combined waveform 506 can represent an increase in the systolic peak and a drop in the diastolic pressure, which can cause various health complications. For example, the change in waveform can impact the workload on the left ventricle and can adversely affect coronary profusion.

In view of the health complications that can be associated with reduced arterial compliance, as described above, it can be desirable in certain patients and/or under certain conditions, to at least partially alter compliance properties of the aorta or other artery or blood vessel, or otherwise alter/control flow therein, in order to improve cardiac and/or other organ health.

### Compliance-Enhancing Devices

The present disclosure relates to systems, devices, and methods for at least partially increasing and/or restoring compliance to a fluid vessel, such as the aorta or other arterial (or venous) blood vessel(s), to provide improved perfusion of the heart muscle and/or other organ(s) of the body. Examples of the present disclosure can include compliant tubular devices configured to channel blood circulation therethrough, such that elastic expansion of the tube during systole can be returned to the circulation during diastole to thereby reduce systolic pressure and/or increase diastolic pressure. For instance, a compliance device in accordance with the present disclosure can include an expandable/elastic fluid channel that expands and stores energy during higher-pressure periods of the cardiac cycle (e.g., during the systolic phase) and contracts/compresses during lower-pressure period (e.g., during the diastolic phase) to return the stored energy to the circulation and increase flow through the channel.

In examples, devices of the present disclosure include elastic/compliant tubes configured to couple to a heart valve area and extend into a heart chamber. For instance, a first end of the device can include an anchoring feature configured to secure the device to a native heart valve or an area within the associated blood vessel. A second end of the device can include a prosthetic heart valve and can be positioned within the heart chamber, wherein the prosthetic valve can replace the native valve. The device can allow blood to flow from the heart chamber through the prosthetic heart valve, through the elastic tube, and to the native blood vessel. The elastic tube can be configured to expand and contract with the cardiac cycle. Thus, the device can increase a length and compliance of the output blood vessel for the heart chamber.

In examples, by disposing compliant implants within native anatomy/blood vessel, as opposed to solutions involving blood vessel grafts and/or resection, incidences of blood leakage and/or rupture of the expandable inner tube can be contained within the target blood vessel, thereby reducing hazards associated with extravascular arterial blood leakage, such as within the abdominal and/or chest cavity. Further, in examples, the compliant devices implants can be delivered through a minimally invasive procedure, which can help prevent complications associated with other types of procedures.

Methods and/or structures disclosed herein for treating a patient also encompass analogous methods and/or structures performed on or placed on a simulated patient, which is useful, for example, for training, for demonstration, for procedure and/or device development, and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof, synthetic, or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

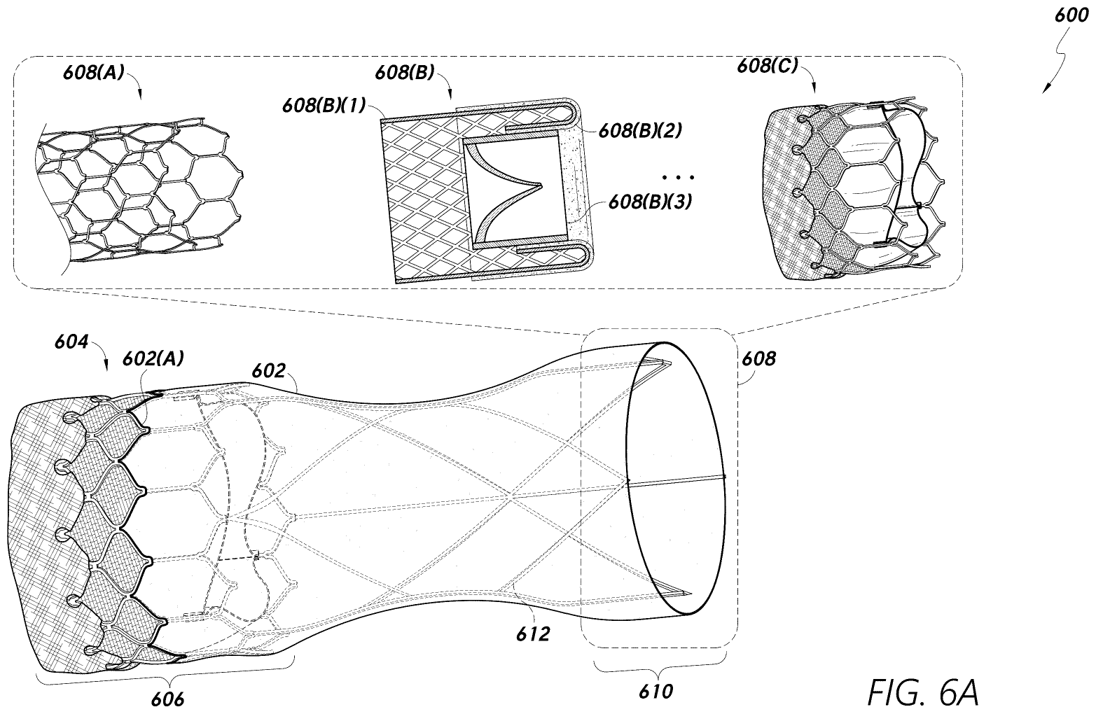

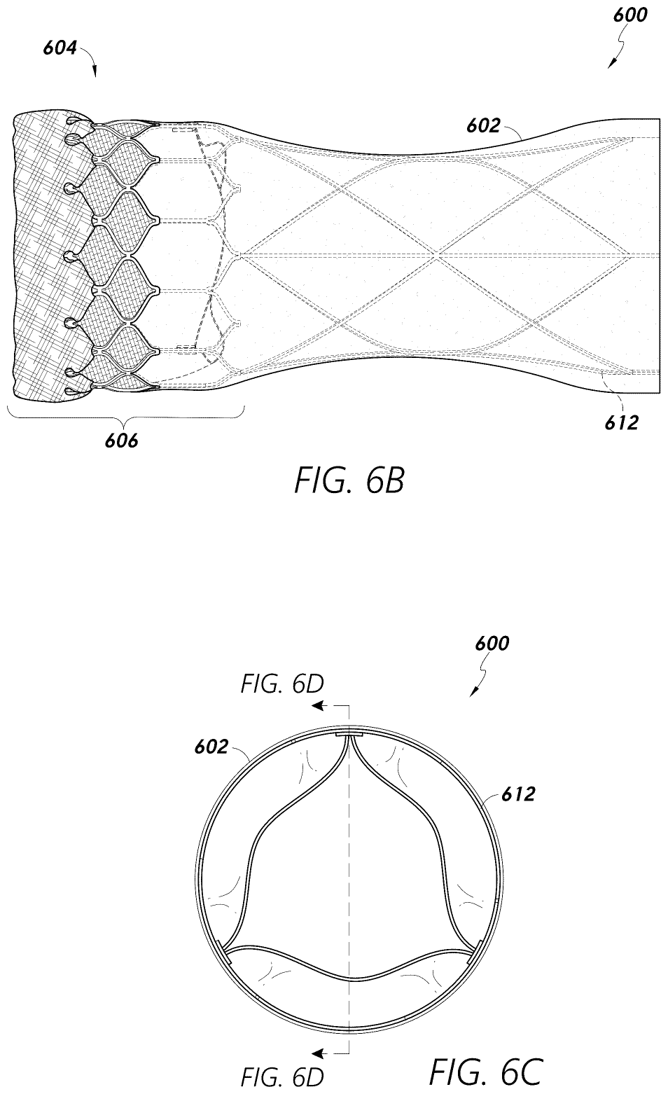

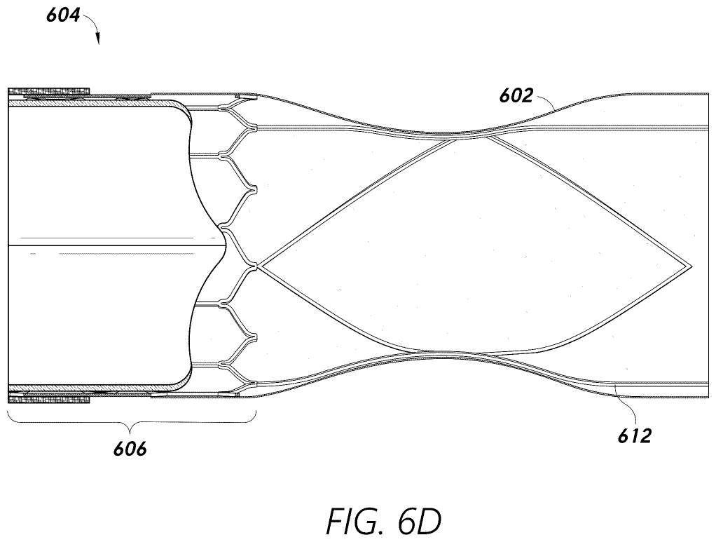

FIGS. 6A, 6B, 6C, and 6D provide perspective, side, front, and cross-sectional views, respectively, of an example device/system 600 that can be configured to enhance compliance of a fluid vessel (also referred to as “the implant device 600”). The device 600 can include an elastic/compliant/flexible/expandable tube/channel/conduit 602 or other structure configured to radially expand and/or contract, such as based on luminal/radial pressure within an inner channel of the elastic tube 602. In this example, the device 600 includes a prosthetic valve(s) 604, such as a prosthetic heart valve, coupled to or integral with a first end/end portion/section 606 of the device 600/elastic tube 602. The first end 604 (also referred to as “the valved portion 606”) can be configured to be disposed within a fluid chamber, such as a heart chamber, or elsewhere. The device 600 can also include a feature/structure/element(s) 608 associated with (e.g., coupled to or integral with a second end/end portion/section 610 of the device 600/elastic tube 602. The feature 608 can be configured to anchor/secure/attach the device 600/elastic tube 602 to the target anatomy and/or configured to facilitate various other functionality. For ease of discussion, the feature 608 will generally be referred to as “the anchoring feature 608” or “the anchor 608.” However, the feature 608 may or may not include an anchoring structure. The device 600 can also include a frame 612 that is generally disposed within the elastic tube 602 and configured to prevent the elastic tube 602 from radially compressing/collapsing. The compliance-enhancement implant device 600 can include one or more one-way valves, such as the prosthetic valve 604, which can be configured and/or oriented to permit flow in a desired flow direction through a channel of the device 600, while restricting or blocking flow in the reverse direction.

The elastic tube 602 (sometimes referred to as “the radially-expandable tube 602”) can be constructed of a compliant material, such as an elastomeric polymer or other material configured to radially expand/stretch and contract/recoil in response to changing pressure/force conditions. For instance, the elastic tube 602 can include a thermoplastic polyurethane (TPU), nylon, etc. In some examples, the elastic tube 602 comprises biological tissue. Further, in some examples, the elastic tube 602 comprises a woven structure, such as a woven memory metal braided structure, or the like. In examples, the elastic tube 602 includes a material to promote tissue growth.

The elastic tube 602 (also referred to as “the sleeve 602” or “tubular structure 602”) can take a cylindrical shape/form or another shape/form when in a non-expanded/default state. That is, the elastic tube 602, in a natural, relaxed, and/or de-pressurized configuration/state can have a straight cylindrical shape/form. In some instances, in a radially-expanded state (pressurized configuration/state), the elastic tube 602 can have an at least partly outwardly-/externally-convex cylindrical shape. The elastic tube 602 can be configured to cycle/change between a relaxed state in low-pressure periods (e.g., diastolic phase of the cardiac cycle), wherein the elastic tube 602 is generally cylindrical or slightly concave or convex in the relaxed state, and a radially expanded state in high-pressure periods (e.g., systolic phase of the cardiac cycle), wherein the elastic tube 602 radially expands/bows-outward. The elastic tube 602 can function as an arterial/blood flow optimizer to generate vascular compliance.

The anchoring feature 608 can be implemented in a variety of manners to anchor/secure/attach the device 600 (also referred to as “the implant device 600”) to the target tissue and/or to provide other functionality. For example, the anchoring feature 608 can anchor the device 600 within a native heart valve, fluid blood vessel, or other tissue. In examples, the anchoring feature 608 can anchor the device 600 to a heart valve area, which can include a heart valve, portion of blood vessel within proximity to the heart valve (e.g., within a predetermined distance), and/or portion of a heart chamber within proximity to the heart valve (e.g., within a predetermined distance).

In examples, the anchoring feature 608 can be implemented as elements that are self-expandable, balloon/device expandable, or otherwise configured to expand or deform to couple to the anatomy. For instance, the anchoring feature 608 can be attached to tissue in a self-expanding/contracting manner. Alternatively, or additionally, the anchoring feature 608 can be manipulated by a device/physician to attach to the tissue (e.g., device/physician expandable). In examples, the device 600 can be configured to be compressed (e.g., radially compressed to a delivery/compressed state) and transported within a delivery catheter/sheath or other tubular delivery system, such as in the case of a minimally invasive procedure through a percutaneous opening or natural orifice. As such, the device 600 can be a percutaneously-placeable implant. Alternatively, the device 600 can be implanted through another type of procedure, such as an open surgery.

In some instances, the anchoring feature 608 include barbs, patches, pins, coils, screws, tabs, hooks, wires, spikes, or other tissue anchor means configured to embed in and/or hold to the associated anatomy. For example, the anchoring feature 608 can have wires or barbs having free ends that project radially outward to puncture the tissue of the native tissue and secure the device 600 in place. In some implementations, such wires/barbs have shape-memory that predisposes such structure to deflect radially outwardly once deployed from a capsule/sheath to facilitate anchoring of the device 600 to the native blood vessel.

Although one anchoring feature 608 is discussed/shown in some examples, the device 600/elastic tube 602 can include any number of anchoring features disposed at any location along the device 600. To illustrate, the anchoring feature 608 or an additional anchoring feature can be positioned closer to the first end 606 of the device 600, at the first end 606, or at another location.

As shown in FIG. 6A, in examples the anchoring feature 608 is implemented as/with a stent/frame structure 608(A). The frame 608(A) can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction.

The frame 608(A) can be formed using any suitable process, such as by stamping or machining the frame structure from a sheet or tube of metal. The frame 608(A) can be made of an at least partially rigid material, such as metal, plastic, etc. For example, the frame 608(A) can comprise stainless steel or nitinol. Where nitinol or other shape-memory metal or material is implemented, the frame 608(A) can be self-expanding. For instance, an expandable stent-type frame can be configured to expand radially from a compressed delivery configuration to the expanded state shown at 608(A). In some implementations, an array of struts is formed from a sheet of metal, which is rolled into a cylinder to form the tubular/cylindrical form of the frame 608(A). In some implementations, a balloon catheter is used to deliver and/or expand the frame 608(A) for securing in a heart valve, blood vessel, or other body cavity.

In some cases, the outside or inside of the frame 608(A) is covered with a fabric, polymer cover, or other material. The covering/cover can promote tissue ingrowth within the inner diameter of the native blood vessel. The cover can be disposed on an outer surface or area of the frame 608(A), and/or can be disposed/applied to the inner diameter of the frame 608(A) on an inside thereof. In some implementations, the cover comprises a cloth or polymer sleeve which can be at least partially elastic, or alternatively nonelastic. The cover can be applied over or within the frame 608(A) in any suitable or desirable manner. For example, the cover can be applied using an electrical or mechanical spinning (e.g., rotary jet spinning, electrospinning, or similar) application process or other deposition process.

As also shown in FIG. 6A, in examples the anchoring feature 608 is implemented as/with a docking station/structure 608(B) (also referred to as “the valve docking station 608(B)”) configured to receive/couple to a prosthetic valve. In examples, the prosthetic valve of the docking station 608(B) can replace a native valve over which the device 600 is implanted. Further, in examples the prosthetic valve of the docking station 608(B) can provide functionality to implement a desired blood flow characteristic, in the case when multiple prosthetic valves are included in on the device 600 or otherwise. For instance, the device 600 can include the prosthetic valve 604 at the first end 606 of the device 600 and a prosthetic valve within the docking station 608(B) at the second end 608 of the device 600. The individual blood flow characteristics of the two prosthetic valves can be tuned/designed/configured to collectively satisfy certain criteria, such as to control blood flow pressure through the prosthetic valves in a cooperative manner. In examples, a flow analysis can be implemented (e.g., simulated or actual) to determine the types of prosthetic valves to implement (e.g., the characteristics of the prosthetic valves). In examples, two prosthetic valves at separate ends of the elastic tube 602 can assist in allowing blood to at least temporarily collect within the elastic tube 602 or to provide other desired results.

In examples, the docking station 608(B) can facilitate a prosthetic valve to be more easily replaced over time, such as in the case where a first prosthetic valve wears out and needs to be replaced with a newer prosthetic valve, where a different prosthetic valve is needed due to different health issues of the patient, and so on. For instance, since patient tissue will generally not grow over the prosthetic valve in the docking station 608(B), such prosthetic valve can be removed more easily from the docking station 608(B) without disrupting the tissue. In some cases, a native valve/leaflet includes one shape/form, while a prosthetic valve/prosthetic leaflet implanted in the docking station 608(B) (or at the first end 606 of the device 600) includes a different shape/form or other characteristics.

The docking station 608(B) can include an outer frame/stent 608(B)(1) configured to secure the docking station 608(B) to the target anatomy (which can be similar to or the same as the frame 608(A), a seat/receiving portion 608(B)(2) configured to receive a prosthetic valve 608(B)(3), any of the features of the frame 608(A), and/or other features. The outer frame 608(B)(1) can be made of any at least partially rigid material, such as metal, plastic, etc. For example, the outer frame 608(B)(1) can comprise stainless steel or nitinol. Where nitinol or other shape-memory metal or material is implemented, the outer frame 608(B)(1) can be self-expanding. For instance, an expandable stent-type frame can be configured to expand radially from a compressed delivery configuration to the expanded state illustrated at 608(B). As shown, the outer frame 608(B)(1) and seat 608(B)(2) can be coupled together to form a channel through a central portion of the docking station 608(B) (where the prosthetic valve 608(B)(3) is located) and/or to prevent blood flow from flowing around the external walls of the outer frame. That is, the docking station 608(B) can seal to the target tissue to create a channel for blood to flow through the prosthetic valve 608(B)(3). In some instances, the outer frame 608(B)(1) is rolled back/bent (e.g., 180 degrees) to form the channel and/or seat 608(B)(2). In examples, the outside or inside of the docking station 608(B) is covered with a fabric, polymer cover, or other material.

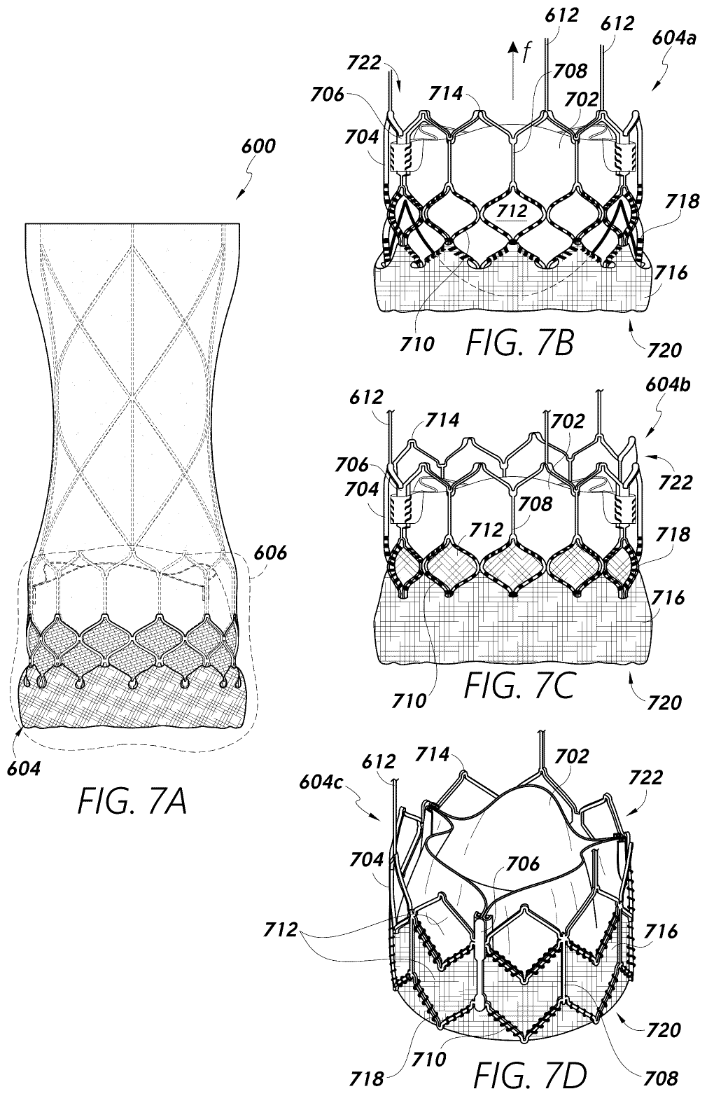

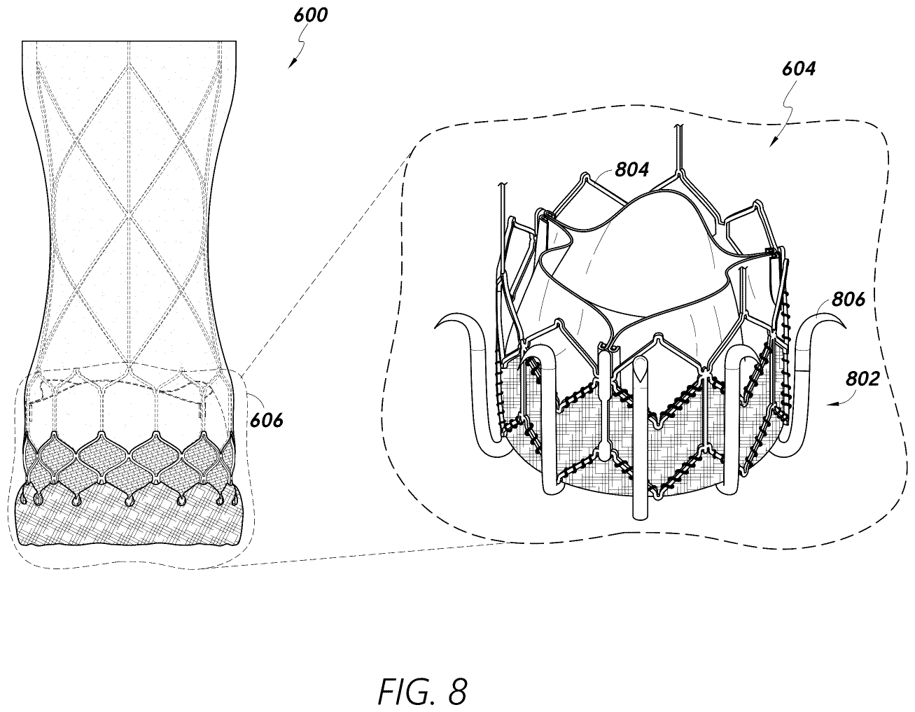

Further, as shown in FIG. 6A, in examples the anchoring feature 608 is implemented as/with another prosthetic valve 608(C). The example prosthetic valve 608(C) can include a variety of forms, such as any of the prosthetic valves discussed herein, including the example prosthetic valves of FIGS. 7B, 7C, and 7D and/or the example prosthetic valve of FIG. 8. In examples, the prosthetic valve 608(C) can include substantially the same/similar dimensions as a native valve in which the device 600 is implanted, since the prosthetic valve 608(C) is not implanted within the native valve. However, the prosthetic valve 608(C) can have other forms/dimensions. In examples, such as when the device 600 is longer than a particular distance (e.g., more than half the length of the ventricle, for instance), the device 600 can include an anchoring structure to anchor to patient tissue, such as that illustrated in FIG. 8, and/or the device 600 can include a cage/protective frame structure around the prosthetic valve 608(C) to prevent anatomy from disrupting the prosthetic valve 608(C).

The frame/wire frame 612 (also referred to as “the frame segment 612,” “the internal frame 612,” or “tube frame 612”) can include a structure comprising a plurality of struts/bars/wires configured to prevent the elastic tube 602 from radially compressing/collapsing. In some instances, such as that shown in FIGS. 6A-6D, the tube frame 612 includes longitudinal/elongate struts that are oriented around a perimeter and cell struts forming one or more cells between the longitudinal struts. The cell struts can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction. In examples, the internal frame 612 can be at least somewhat convex to assist in causing the elastic tube 602 to expand, instead of compressing (e.g., during systole). Although the internal frame 612 is discussed in many examples as being positioned within the elastic tube 602, the internal frame 612 can be positioned outside/external to the elastic tube 602.

The internal frame 612 can be made of any at least partially rigid material, such as metal, plastic, etc. For example, the internal frame 612 can comprise stainless steel or nitinol. Where nitinol or other shape-memory metal or material is implemented, the internal frame 612 can be self-expanding. For instance, an expandable stent-type frame can be configured to expand radially from a compressed delivery configuration to the expanded state shown. In some implementations, the internal frame 612 is formed from a sheet of metal, which is rolled into a cylinder to form the tubular/cylindrical form of the internal frame 612. In examples, the internal frame 612 can be implemented as a self-expandable, balloon/device expandable, or otherwise expandable frame. The internal frame 612 can be formed using any suitable process, such as by stamping or machining the frame structure from a sheet or tube of metal.

In some cases, the outside or inside of the internal frame 612 is covered with a fabric, polymer cover, or other material. The cover can be disposed on an outer surface or area of the internal frame 612, and/or can be disposed/applied to the inner diameter of the internal frame 612 on an inside thereof. In some implementations, the cover comprises a cloth or polymer sleeve which can be at least partially elastic, or alternatively nonelastic. The cover can be applied over or within the internal frame 612 in any suitable or desirable manner. For example, the cover can be applied using an electrical or mechanical spinning (e.g., rotary jet spinning, electrospinning, or similar) application process or other deposition process. In examples, the cover can be implemented in addition to the elastic tube 602, which cover can help prevent the elastic tube 602 from radially compressing (e.g., through openings in the cells).

In some instances, the internal frame 612 includes a relatively small number of struts/wires (in comparison to a threshold or typical stent). For example, the internal frame 612 can include a few longitudinal struts and a few cell struts to hold the longitudinal struts in place, as shown in FIGS. 6A-6D. In examples, the internal frame 612 can include less struts than a frame of the anchoring feature 608, since the internal frame 612 may not (in some instances) contact the anatomy in the patient or otherwise be subjected to external forces, like the anchoring feature 608. To illustrate, the internal frame 612 can include sufficient strut structure to prevent the elastic tube 602 from deflating or otherwise falling into the central axial channel of the device 600, which may be less strut structure than that needed to anchor a stent to a target anatomy.

In the examples shown, the elastic tube 602 extends over the internal frame 602 and a portion of the prosthetic valve 604 and to provide a sealed path for blood flow through the device 600. Here, the elastic tube 602 is attached/coupled to the frame/cover of the prosthetic valve 604 at 602(A); however, the elastic tube 602 can be attached/coupled at other locations and/or in other manners to seal the internal channel of the device 600. The elastic tube 602 and the prosthetic valve 604 can be attached/coupled together in a variety of manners. In some cases, the elastic tube 602 extends completely over the prosthetic valve 604 to the inflow end of the device 600 and/or extends all the way to the outflow end of the device 600 (e.g., where the feature 608 is located). The elastic tube 602 can be disposed around or inside at least a portion of the prosthetic valve 604 and/or the internal frame 612.

In examples, at least a portion of the device 600 includes a concave form (or convex, in some cases), as shown in the example of FIGS. 6A-6D. Here, the internal frame 612 and/or the elastic tube 602 (and/or the valved portion 606) is at least partially concave such that a medial portion of the internal frame 612 and/or the elastic tube 602 has a smaller diameter than one or more ends/end portions of the internal frame 612 and/or the elastic tube 602. This can minimize an amount of volume/space that the device 600 takes up within the implanted chamber, such as the left ventricle, and/or maximize the amount of change in volume that the device 600 can provide. For instance, the medial section of the elastic tube 602 can be configured to expand from a concave form (in a non-pressurized state) to a straight or convex form (in a pressurized state). However, in some cases the internal frame 612 and/or the elastic tube 602 does not include a concave form. Further, in some examples, the medial portion of the elastic tube 602 is more elastic than one or more end portions of the elastic tube 602.

FIGS. 7B, 7C, and 7D illustrate example prosthetic valves that can be implemented with the implant device 600 in accordance with one or more examples. Here, the example prosthetic valves are discussed and illustrated in the context of the prosthetic valve 604 (the valved portion 606) of the device 600, as shown in FIG. 7A and elsewhere. However, the example prosthetic valves can additionally, or alternatively, be implemented at other ends of the device 600, such as at the anchoring feature 608 side of the device 600. In some implementations, the device 600 and/or the prosthetic valve 604 can be mechanically expanded or radially self-expand from a compressed delivery state to the operational state under its own resiliency when released from a delivery system. In examples, the prosthetic valve 604 can be similar in one or more respects to the Edwards Lifesciences SAPIEN XT™, SAPIEN 3™, and/or SAPIEN 3 Ultra™ transcatheter prosthetic heart valves.

The prosthetic valve 604 can include valve leaflets/structure/feature(s) 702 supported inside a frame/frame structure/segment 704 (also referred to as “the valve frame 704”). The valve leaflets 702 can function by opening to permit flow in the presence of a pressure gradient in the direction of the prosthetic valve 604, such that the prosthetic valve 604 opens to permit flow and closes with each cardiac cycle to prevent flow in the opposite direction. The valve leaflets 702 can comprise any suitable or desirable material(s), such as biological tissue, polymer materials, etc. The prosthetic valve 604 can comprise any suitable or desirable number of leaflets, such as three, as shown in the illustrated examples. Although the prosthetic valve 604 is illustrated as including valve leaflets 702, other types of flow-control mechanisms can be utilized to achieve a desired direction and/or rate of flow through the device 600 in accordance with aspects of the present disclosure.

The valve frame 704 can include an annular structure having a plurality of vertically extending commissure attachment posts 706, which attach and/or help shape the leaflet structure 702 therein. Additional vertical posts or strut members 708, along with circumferentially extending strut members 710 can help form the rest of the valve frame 704. In some examples, the struts 708 and/or 710 can form axial rows of cells 712, which can be circumferentially staggered/offset, or the rows of cells 712 can be generally circumferentially aligned. The cells 712 can have any suitable or desirable shape (e.g., oval/ellipse, diamond/rhombus, hexagonal diamond/polygon, etc.). The open cell geometry of the valve frame 704 can facilitate coronary access, in some cases. With further reference to the example of FIG. 7D, the strut members 708/710 can form chevron/zig-zag shapes/cells in a base portion of the valve frame 704. The struts 708/710 can form edged crown portions 714 or apices at the inflow and/or outflow ends of the valve frame 704.

The valve frame 704 can be made of any at least partially rigid material, such as metal, plastic, etc. For example, the valve frame 704 can comprise stainless steel or nitinol. Where nitinol or other shape-memory metal or material is implemented, the valve frame 704 can be self-expanding. For instance, an expandable stent-type frame can be configured to expand radially from a compressed delivery configuration to the expanded state shown. In some implementations, an array of struts is formed from a sheet of metal, which is rolled into a cylinder to form the tubular/cylindrical form of the valve frame 704. The valve frame 704 can be formed using any suitable process, such as by stamping or machining the frame structure from a sheet or tube of metal. In some implementations, the valve frame 704 can be plastically expandable to its functional size by a balloon or another expansion device, in which case the frame 704 can be made of a plastically expandable material, such as stainless steel or a cobalt chromium alloy. Other suitable materials can also be used.

As described above, the valve frame 704 can be designed to be radially crimped or compressed to facilitate endovascular delivery to the target implant site. For example, the valved portion 606 can be positioned at a native valve annulus (e.g., aortic valve annulus), where the valve frame 704 can be expanded to an operational state, for example, by an expansion balloon, such that the leaflet structure 702 or other flow-control mechanism of the valved portion 606 regulates blood flow through the native valve annulus.

In some implementations, the valve frame 704 is at least partially covered within and/or without by a sealing skirt 716, which can comprise any suitable or desirable material, such as textured polyethylene terephthalate (PET). The skirt 716 can be attached to an inner surface of the valve frame 704 to form a suitable/desirable attachment surface for the valve leaflets 702. The skirt 716 can be attached to the valve frame 704 in any suitable or desirable manner, such as through use of adhesive or other attachment means. In some examples, the skirt 716 is attached to an inner and/or outer surface of the valve frame 704 via one or more sutures 718, which can be wrapped around the various struts of the valve frame 704. The skirt 716 can provide a relatively more substantive attachment surface for portions of the leaflet structure 702 positioned closer to an inflow end 720 of the device 600.

In some examples, as shown in FIGS. 7B and 7C, the skirt 716 can be folded over the inflow end 720 of the frame 704 to cover over an outer diameter/surface of the frame 704. The portion of the skirt 716 on the outside of the frame 704 can facilitate attachment to the patient anatomy, such as through tissue ingrowth and/or frictional fit between the frame 704 and the anatomy (e.g., aortic valve annulus, aorta, etc.). In examples, the elastic tube 602 can be coupled/attached to the skirt 716 to seal the elastic tube 602 to the device 600 and provide a sealed channel for fluid/blood flow through the device 600. However, the elastic tube 602 can be attached to the prosthetic valve 604 in other manners.

At an outflow end 722, the internal frame 612 can extend from the prosthetic valve 604, as shown in FIGS. 7B, 7C, and 7D. For example, the frame 704 of the prosthetic valve 604 can be coupled to or integral with the internal frame 612, such as coupled via struts/elements 706, 708, 710, 714, etc. The elastic tube 602 (not shown in the right-side images of FIGS. 7B, 7C, and 7D) can extend over at least a portion of the frame 704 of the prosthetic valve 604 and attach to the frame 704, the skirt 716, and/or other portions of the prosthetic valve 604.

As noted above, although discussed in the context of the prosthetic valve 604, any of the example prosthetic valves of FIGS. 7B, 7C, and 7D can additionally, or alternatively, be implemented at the second end 610 of the device 600 and/or elsewhere on the device 600.

FIG. 8 illustrates another example prosthetic valve that can be implemented with an implant device in accordance with one or more examples. Here, the example prosthetic valve is discussed and illustrated in the context of the prosthetic valve 604 (the valved portion 606) of the device 600. However, the example prosthetic valve can additionally, or alternatively, be implemented at other ends of the device 600, such as at the anchoring feature 608 side of the device 600. As similarly discussed above, in some implementations, the device 600 and/or the prosthetic valve 604 can be mechanically expanded or radially self-expand from a compressed delivery state to the operational state under its own resiliency when released from a delivery system. In examples, the prosthetic valve 604 can be similar in one or more respects to the Edwards Lifesciences SAPIEN XT™, SAPIEN 3™, SAPIEN 3 Ultra™, and/or Evoque transcatheter prosthetic heart valves.

As shown in FIG. 8, the prosthetic valve 604 can include one or more arms/elongate members 802 that are coupled to a frame 804 of the prosthetic valve 604 (which can be similar to or the same as the frame 704 discussed in reference to FIGS. 7B, 7C, and 7D). The one or more arms 802 can be distributed circumferentially around the frame 804 and/or attached to an inflow end of the prosthetic valve 604. The one or more arms 802 can initially project/extend from the frame 804 radially and then project/extend upwards/longitudinally (relative to FIG. 8) towards an outflow end of the prosthetic valve 604, as shown. At the end of each of the one or more arms 802, the respective arm 802 can include an anchoring feature 806 configured to anchor/secure the prosthetic valve 604 to patient anatomy, such as tissue/wall of a heart chamber (when the prosthetic valve 604 is positioned within the heart chamber). The anchoring feature(s) 806 can include a barb(s), patch(es), pin(s), coil(s), screw(s), tab(s), hook(s), wire(s), spike(s), or other tissue anchor means configured to embed in and/or hold to the anatomy.

In examples, the one or more arms 802 and/or the one or more anchoring features 806 can assist in holding/maintaining the prosthetic valve 604 in a relatively secure manner, such as to prevent the prosthetic valve 604 from moving around within a heart chamber. In examples, the one or more arms 802 and/or the one or more anchoring features 806 can be configured to mechanically expanded or radially self-expand from a compressed delivery state to the operational state shown in FIG. 8. The one or more arms 802 and/or the one or more anchoring features 806 can implement an anchoring structure to anchor the prosthetic valve 604 to target tissue, such as tissue within a heart chamber or other tissue.

As noted above, although discussed in the context of the prosthetic valve 604, the example prosthetic valve of FIG. 8 can additionally, or alternatively, be implemented at the second end 610 of the device 600 and/or elsewhere on the device 600.

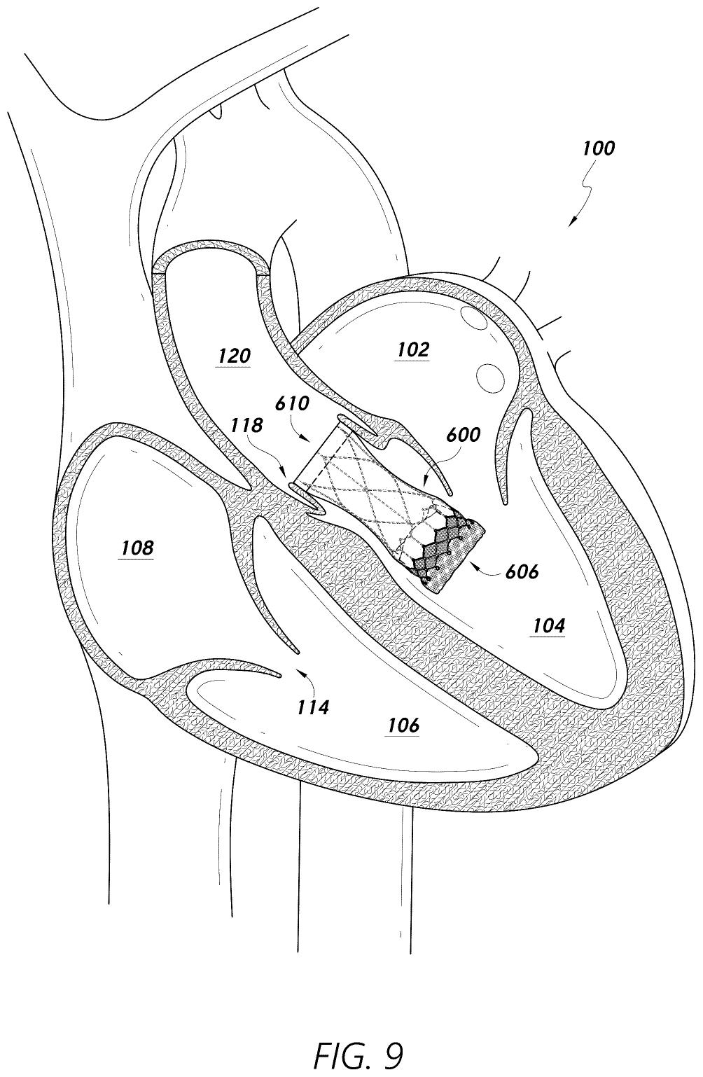

FIG. 9 illustrates the device 600 disposed/implanted within example anatomy of a patient, namely the aortic valve 118 and left ventricle 104 in a cross-sectional view of the heart 100, in accordance with one or more examples. In this example, the second end/end portion 610 of the device 600 is implanted at the aortic valve 118 (e.g., within the annulus of the aortic valve 118), with the leaflets of the aortic valve 118 displaced to the sides to make room for the device 600. However, the second end 610 can be implanted at other locations, such as within the aorta 120 near the aortic valve 118, within the left ventricle 104, or elsewhere. The device 600 can be positioned/deployed such that the first end/end portion 606 of the device 600 is positioned/disposed within the left ventricle 104, as shown. Here, the first end 606 is positioned upstream and the second end 610 is positioned downstream relative to the flow of blood through the heart 100.

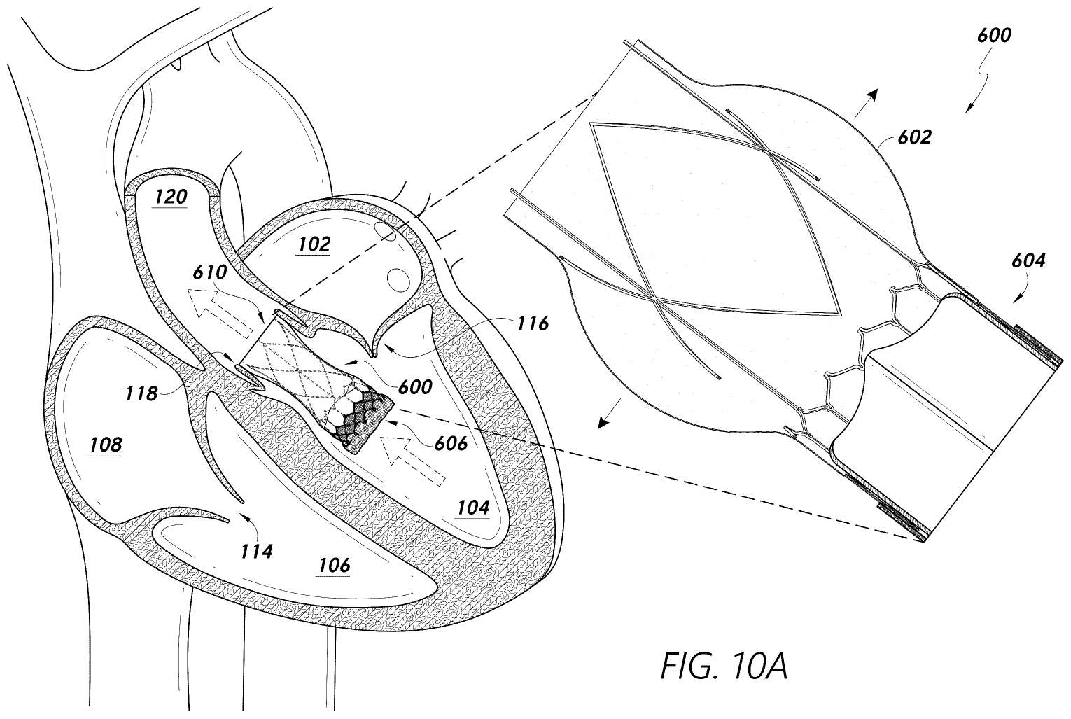

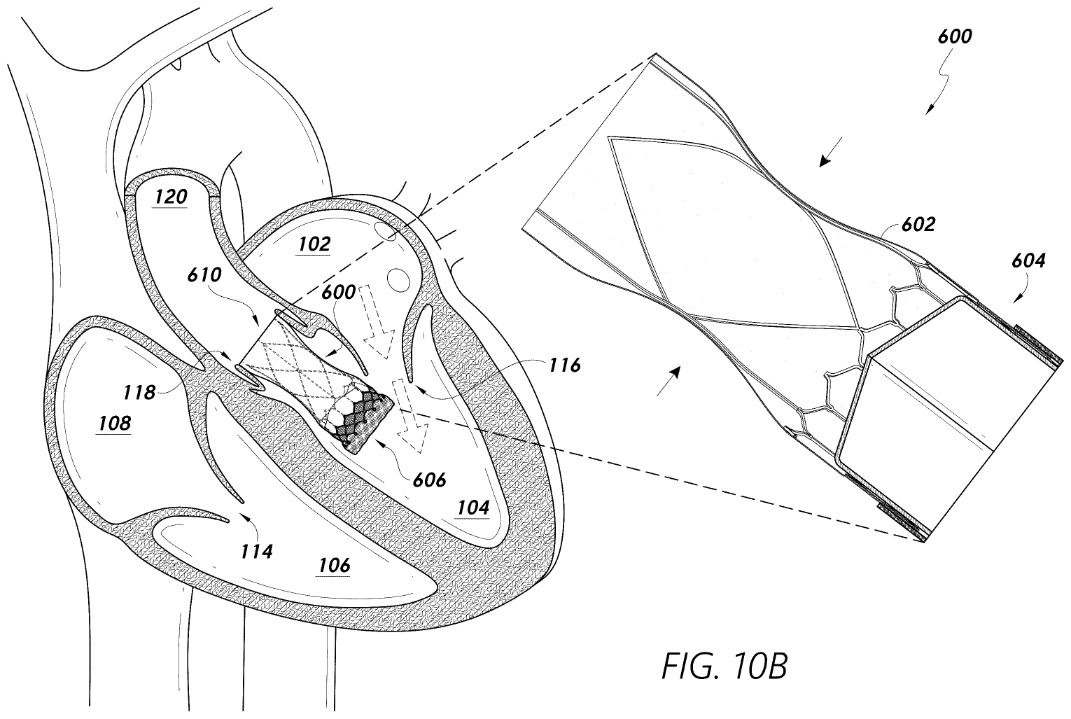

FIGS. 10A and 10B illustrate operation of the device 600 as implanted in the aortic valve 118 and left ventricle 104 during various phases of the cardiac cycle in accordance with one or more examples. FIG. 10A depicts expansion of the device 600 during at least a portion of systole, while FIG. 10B depicts contraction of the device 600 during at least a portion of diastole.

As shown in FIG. 10A, during systole, the mitral valve 116 is closed and the prosthetic valve 604 of the device 600 (which is configured to replace the functionality of the aortic valve 118) is open to allow blood flow through the device 600 and to the aorta 120, such as through an internal channel within the elastic tube 602. As blood is forced into the device 600, the blood/luminal pressure within the elastic tube 602 increases and the elastic tube 602 expands, thereby storing blood and energy within the elastic tube 602 in the form of elastic stretch. In examples, the internal frame 612 of the device 600 can assist in preventing the elastic tube 602 from compressing inward during systole or at other times.

In contrast, as shown in FIG. 10B, during diastole, the mitral valve 116 is open and the prosthetic valve 604 of the device 600 is closed to allow blood to flow into the left ventricle 104 from the left atrium 102. Here, the blood/luminal pressure within the elastic tube 602 decreases, resulting in the elastic tube 602 radially compressing and releasing the blood into circulation within the aorta 120. That is, the stored energy in the elastic tube 602 causes the elastic tube 602 to contract/recoil back towards the original state thereof due to decreasing blood pressure within the device 600.

Thus, as with other examples disclosed herein, the device 600 can include an axial internal central fluid channel within the elastic tube 602 that is configured to radially expand and retract/recoil based on changing pressure conditions within the channel (e.g., changing luminal pressures). As the elastic tube 602 radially expands and contracts, the volume of the channel increases and decreases in a manner to provide compliance for the blood vessel in which it is implanted, such as the aorta 120 in this example.

Although the device 600 is illustrated with a single prosthetic valve in the examples of FIGS. 9, 10A, and 10B (and elsewhere), the device 600 can include additional prosthetic valves, such as one positioned at the aortic valve 118 (at the second end 610 of the device 600), as discussed herein.

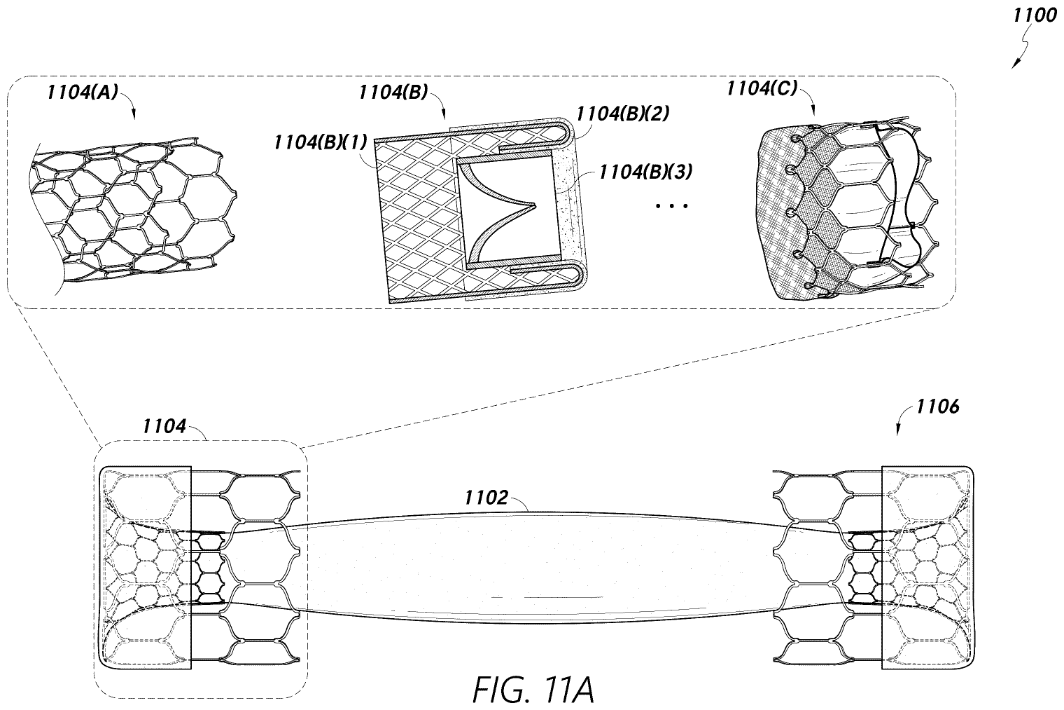

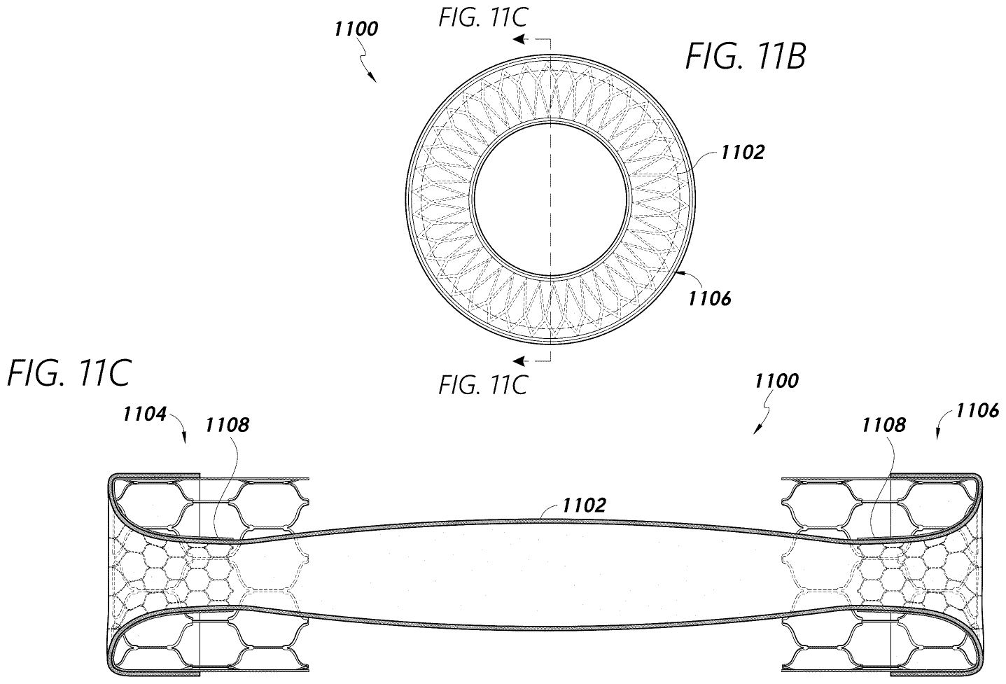

FIGS. 11A, 11B, and 11C provide side, front, and cross-sectional views, respectively, of another example device/system 1100 that can be configured to enhance compliance of a fluid vessel (also referred to as “the implant device 1100”). The device 1100 can include an elastic/compliant/flexible/expansive tube/channel/conduit 1102 or other structure configured to radially expand and/or contract, such as based on luminal/radial pressure within an inner channel of the elastic tube 1102. In this example, the device 1100 includes a first anchoring feature/structure/element 1104 coupled to or integral with a first end/end portion/section of the device 1100/elastic tube 1102 and a second anchoring feature/structure/element 1106 coupled to or integral with a second end/end portion/section of the device 1100/elastic tube 1102. The anchoring features 1104, 1106 can be configured to anchor/secure/attach the device 1100/elastic tube 1102 to the target anatomy and/or configured to facilitate other functionality. The elastic tube 1102 can be coupled between the anchoring features 1104, 1106, as shown, to provide a channel through the implant device 1100/anchoring feature 1104/1106. In examples, the elastic tube 1102 can be similar to the elastic tube 602 of the device 600 and/or the anchoring features 1104, 1106 can be similar to the anchoring feature 608/prosthetic valve 604 of the device 600.

The elastic tube 1102 can be constructed of a compliant material, such as an elastomeric polymer or other material configured to radially expand/stretch and contract/recoil in response to changing pressure/force conditions. For instance, the elastic tube 1102 can include a thermoplastic polyurethane (TPU), nylon, etc. In some examples, the elastic tube 1102 comprises biological tissue. Further, in some examples, the elastic tube 1102 comprises a woven structure, such as a woven memory metal braided structure, or the like. In examples, the elastic tube 1102 includes a material to promote tissue growth. The elastic tube 1102 can function as an arterial/blood flow optimizer to generate vascular compliance.

The elastic tube 1102 (also referred to as “the sleeve 1102” or “tubular structure 1102”) can take a cylindrical shape/form or another shape/form, such as a partially concave form, when in a non-expanded/default state. For instance, the elastic tube 1102, in a natural, relaxed, and/or de-pressurized configuration/state can have a straight cylindrical shape/form. In some instances, in a radially-expanded, pressurized configuration/state, the elastic tube 1102 can have an at least partly outwardly-/externally-convex cylindrical shape. The elastic tube 1102 can be configured to cycle between a relaxed state in low-pressure periods (e.g., diastolic phase of the cardiac cycle), wherein the elastic tube 1102 is generally cylindrical or slightly concave or convex, and a radially expanded state in high-pressure periods (e.g., systolic phase of the cardiac cycle), wherein the elastic tube 1102 radially expands/bows-outward. The elastic tube 1102 can be designed/configured to have an outer diameter that is smaller than an inner diameter of a fluid vessel in which the elastic tube 1102 is implanted.

The anchoring feature 1104/1106 can be implemented in a variety of manners to anchor/secure/attach the device 1100 to the target tissue and/or to provide other functionality. For example, the anchoring feature 1104/1106 can anchor the device 1100 within a fluid blood vessel or other tissue, such as a tubular shaped chamber. In examples, the anchoring feature 1104/1106 can anchor at least a portion of the device 1100 to a heart valve area.

In examples, the anchoring feature 1104/1106 can be implemented as elements that are self-expandable, balloon/device expandable, or otherwise configured to expand or deform to couple to the anatomy. For instance, the anchoring feature 1104/1106 can be attached to tissue in a self-expanding/contracting manner. Alternatively, or additionally, the anchoring feature 1104/1106 can be manipulated by a device/physician to attach to the tissue (e.g., device/physician expandable). In examples, the device 1100 can be configured to be compressed (e.g., radially compressed to a delivery/compressed state) and transported within a delivery catheter/sheath or other tubular delivery system, such as in the case of a minimally invasive procedure through a percutaneous opening or natural orifice. As such, the device 1100 can be a percutaneously-placeable implant. Alternatively, the device 1100 can be implanted through another type of procedure, such as an open surgery.

In some instances, the anchoring feature 1104/1106 includes barbs, patches, pins, coils, screws, tabs, hooks, wires, spikes, or other tissue anchor means configured to embed in and/or hold to the associated anatomy. For example, the anchoring feature 1104/1106 can have wires or barbs having free ends that project radially outward to puncture the tissue of the native tissue and secure the device 1100 in place. In some implementations, such wires/barbs have shape-memory that predisposes such structure to deflect radially outwardly once deployed from a capsule/sheath to facilitate anchoring of the device 1100 to the native blood vessel. Although two anchoring features 1104, 1106 are discussed/shown in many examples, the device 1100/elastic tube 1102 can include any number of anchoring features disposed at any location along the device 1100.

As shown in FIG. 11A, in examples the anchoring feature 1104 (which can be representative of the anchoring feature 1104 or the anchoring feature 1106) is implemented as/with a stent/frame structure 1108(A). The frame 1104(A) can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction.

The frame 1104(A) can be formed using any suitable process, such as by stamping or machining the frame structure from a sheet or tube of metal. The frame 1104(A) can be made of any at least partially rigid material, such as metal, plastic, etc. For example, the frame 1104(A) can comprise stainless steel or nitinol. Where nitinol or other shape-memory metal or material is implemented, the frame 1104(A) can be self-expanding. For instance, an expandable stent-type frame can be configured to expand radially from a compressed delivery configuration to the expanded state shown at 1104(A). In some implementations, an array of struts is formed from a sheet of metal, which is rolled into a cylinder to form the tubular/cylindrical form of the frame 1104(A). In some implementations, a balloon catheter is used to deliver and/or expand the frame 1104(A) for securing in a heart valve, blood vessel, or other body cavity.

In some cases, the outside or inside of the frame 1104(A) is covered with a fabric, polymer cover, or other material. The covering/cover can promote tissue ingrowth within the native blood vessel. The cover can be disposed on an outer surface or area of the frame 1104(A) and/or can be disposed/applied to the inner diameter of the frame 1104(A). In some implementations, the cover comprises a cloth or polymer sleeve which can be at least partially elastic, or alternatively nonelastic. The cover can be applied over or within the frame 1104(A) in any suitable or desirable manner. For example, the cover can be applied using an electrical or mechanical spinning (e.g., rotary jet spinning, electrospinning, or similar) application process or other deposition process.

As also shown in FIG. 11A, in examples the anchoring feature 1104/1106 is implemented as/with a docking station/structure 1104(B) (also referred to as “the valve docking station 1104(B)”) configured to receive/couple to a prosthetic valve. In examples, the prosthetic valve of the docking station 1104(B) can replace a native valve over which the device 1100 is implanted. Further, in examples the prosthetic valve of the docking station 1104(B) can provide functionality to implement a desired blood flow characteristic, in the case when multiple prosthetic valves are included in on the device 1100. For instance, the anchoring feature 1104 can include a first prosthetic valve and the anchoring feature 1106 can include a second prosthetic valve, which can be the same or different and/or can be configured to implement particular blood flow characteristics, as similarly discussed above. In examples, the docking station 1104(B) can facilitate a prosthetic valve to be more easily replaced.

The docking station 1104(B) can include an outer frame/stent 1104(B)(1) configured to secure the docking station 1104(B) to the target anatomy (which can be similar to or the same as the frame 1104(A)), a seat/receiving portion 1104(B)(2) configured to receive a prosthetic valve 1104(B)(3), any of the features of the frame 1104(A), and/or other features. The outer frame 1104(B)(1) can be made of any at least partially rigid material, such as metal, plastic, etc. For example, the outer frame 1104(B)(1) can comprise stainless steel or nitinol. Where nitinol or other shape-memory metal or material is implemented, the outer frame 1104(B)(1) can be self-expanding. For instance, an expandable stent-type frame can be configured to expand radially from a compressed delivery configuration to the expanded state illustrated at 1104(B). As shown, the outer frame 1104(B)(1) and seat 1104(B)(2) can be coupled together to form a channel through a central portion of the docking station 1104(B) (where the prosthetic valve 1104(B)(3) is located) and/or to prevent blood flow from flowing around the external walls of the outer frame 1104(B)(1). That is, the docking station 1104(B) can seal to the target tissue to create a channel for blood to flow through the prosthetic valve 1104(B)(3). In some instances, the outer frame 1104(B)(1) is rolled back/bent to form the channel and/or seat 1104(B)(2). In examples, the outside or inside of the docking station 1104(B) is covered with a fabric, polymer cover, or other material.

Further, as shown in FIG. 11A, in examples the anchoring feature 1104/1106 is implemented as/with another prosthetic valve 1104(C). The example prosthetic valve 1104(C) can include a variety of forms, such as any of the prosthetic valves discussed herein. In examples, the prosthetic valve 1104(C) can include substantially the same/similar dimensions as a native valve in which the device 1100 is implanted. Whether one or multiple prosthetic valves are implemented with the device 1100, the prosthetic valve(s) can be configured to permit fluid flow in one direction and prevent fluid flow in the opposite direction.

In examples, as shown in the cross-sectional view of FIG. 11C and elsewhere, the anchoring feature 1104/1106 can form a connection/attachment/frame feature/structure 1108 (also referred to as “the inner frame structure 1108”) for the elastic tube 1102 to attach/couple to the anchoring feature 1104/1106. For instance, the frame of the anchoring feature 1104/1106 can bend back/fold to form a half-torus shaped structure/end 1108, which can act as a sealing portion to a blood vessel so that fluid will flow through just the internal channel of the device 1102. The elastic tube 1102 can attach to the inner frame structure 1108 and/or extend from one end of the device 1100 (e.g., out and/or around the half-torus structure at one end) to the other end of the device 1100 (e.g., out and/or around the half-torus structure at the other end of the device 1100), as shown. For instance, the elastic tube 1102 can extend from the inner channel of the device 1100 around end portions of the device 1100 to an external structure/surface of the device 1100 (e.g., extend from a structure that forms an internal diameter to a structure that forms an outer diameter. In some instances, the elastic tube 1102 can attach to a covering of anchoring feature 1104/1106. In examples, the inner frame structure 1108 has a smaller diameter than an outer diameter of the anchoring feature 1104/1106. In examples, the elastic tube 1102 can have the same diameter as the connection feature 1108 when the elastic tube 1102 is in a non-expanded state. The inner frame structure 1108 can include or be formed in a similar manner as the anchoring feature 1104/1106. The inner frame structure 1108 can be integral with or attached/coupled to the anchoring feature 1104/1106. In examples, such as that illustrated, the inner frame structure 1108 can have a smaller cell size than the anchoring feature 1104/1106.

In some instances, the internal frame structure 1108 extends from the anchoring feature 1104 to the anchoring feature 1106 (and/or is disposed within the elastic tube 1102) to form an internal structure that prevents the elastic tube 1102 from radially compressing/collapsing. Such structure can be similar to the internal frame 612 of the device 600. The internal frame 1108 can include any number of struts/wires.

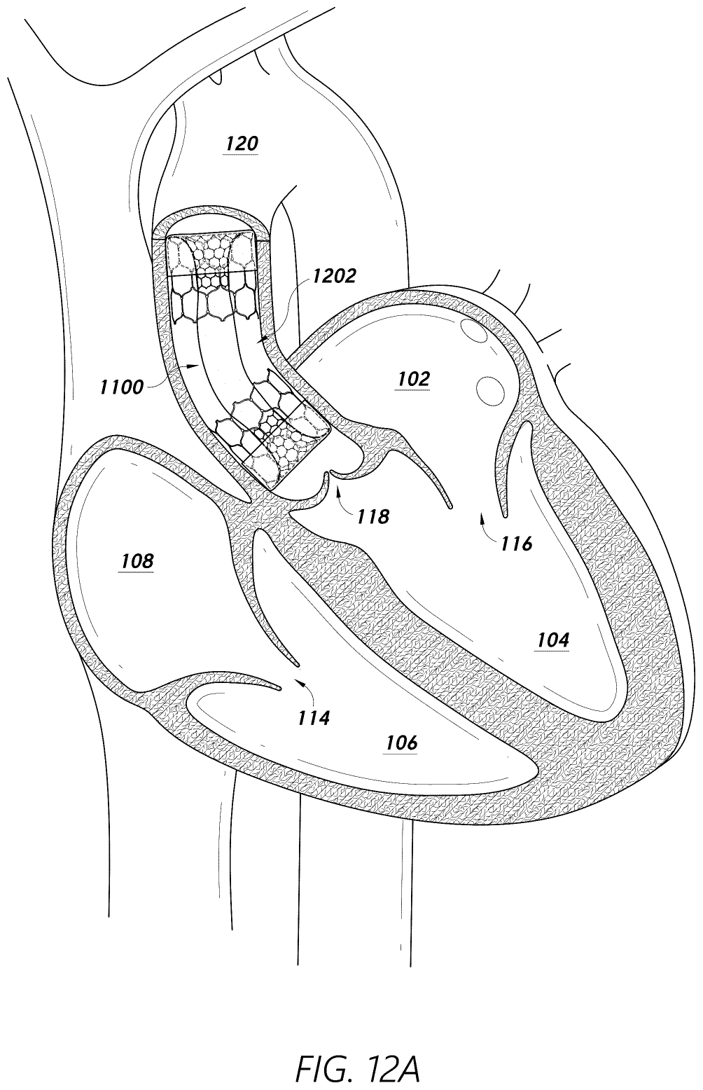

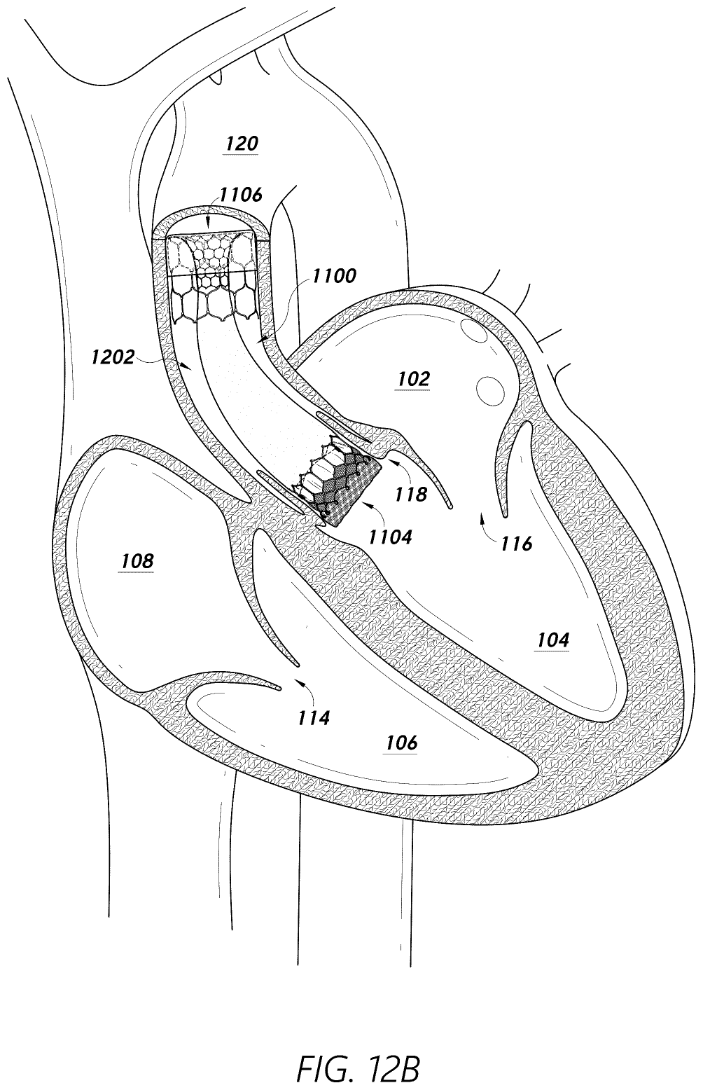

FIGS. 12A and 12B illustrate the device 1100 disposed/implanted within example anatomy of a patient (with a cross-sectional view of the heart 100) in accordance with one or more examples. In FIG. 12A, the device 1100 is implanted within the aorta 120, such as the ascending aorta, within proximity to the aortic valve 118. However, the device 1100 can be implanted at other locations, such as at other locations within the aorta 120 and/or other fluid vessels. Here, the device 1100 is implanted at a position that avoids blocking a passage to another fluid vessel, such as the coronary sinuses. However, the device 1100 can include openings/passage structures within the elastic tube 1102 and/or frame of the device 1100 to allow blood flow to other blood vessels.

In FIG. 12B, the device 1100 is implanted at least partially within the aortic valve 118 (e.g., within the annulus of the aortic valve 118). In particular, the first anchoring feature 1104, which includes a prosthetic valve in this example to replace the native aortic valve 118, is implanted at the aortic valve 118, causing the leaflets of the aortic valve 118 to be displaced to the sides. The device 1100 can be positioned/implanted such that the anchoring feature 1106 is positioned/disposed at least partially within the ascending aorta 120, as shown.

In some examples implementations of FIG. 12A and FIG. 12B, the device 1100 can be implanted in a manner that prevents/minimizes blood flow in an area 1202 in the aorta 120 between the elastic tube 1102 and the inner wall of the aorta 120. For example, while implanting the anchoring features 1104 and 1106 the blood in the area 1202 can be removed and/or the area 1202 can be filled with another fluid, such as by inflating a balloon positioned within the elastic tube 1102 to force blood out of the area 1202, providing suction to remove the blood from the area 1202, and/or disposing/injecting fluid within the area 1202. This can prevent blood from remaining in the area 1202, which can lead to undesirable outcomes.

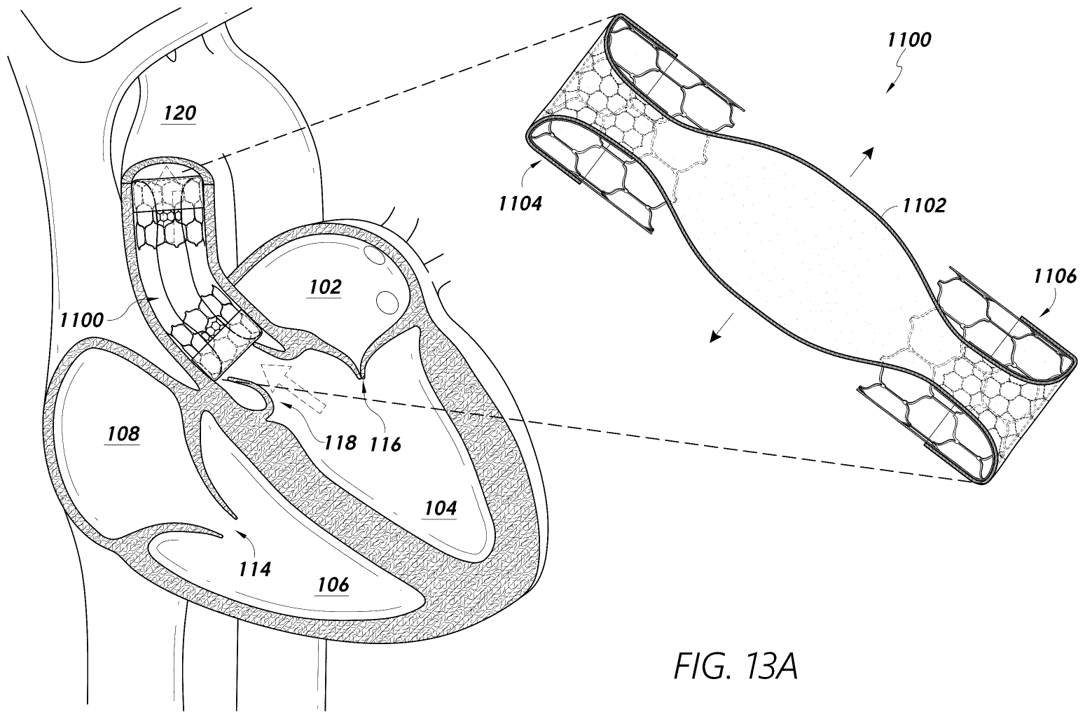

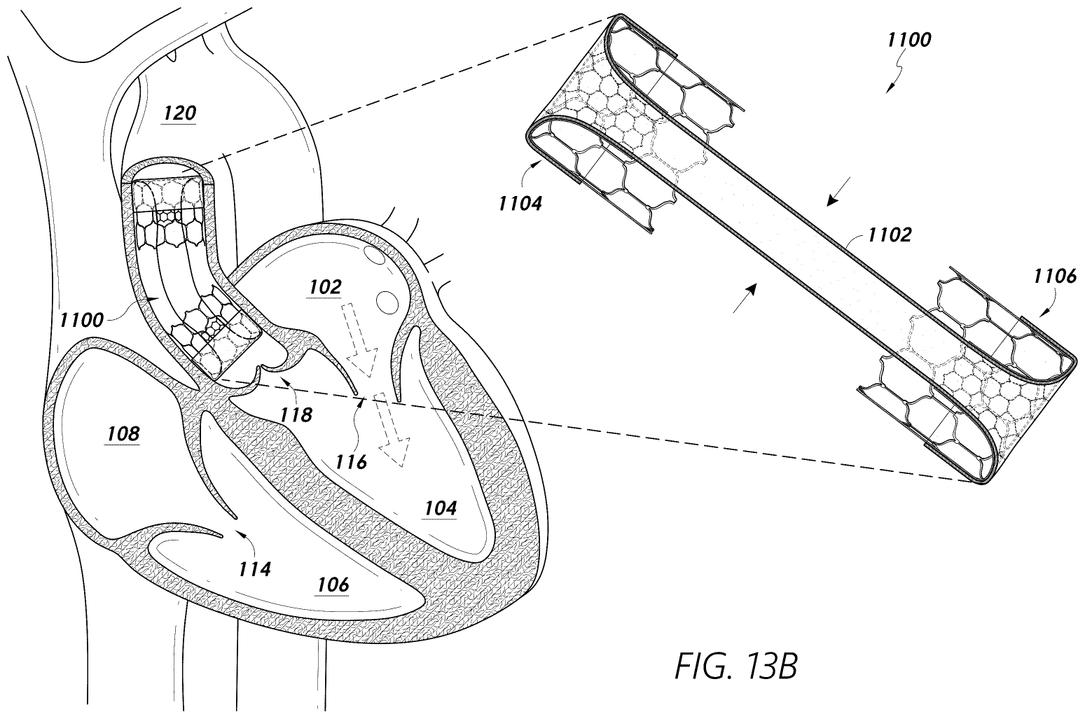

FIGS. 13A and 13B illustrate operation of the device 1100 as implanted in the aorta 120 during various phases of the cardiac cycle in accordance with one or more examples. FIG. 13A depicts expansion of the device 1100 during at least a portion of systole, while FIG. 13B depicts contraction of the device 1100 during at least a portion of diastole. Although the device 1100 is illustrate as being disposed within the aorta 120, the device 1100 can be implanted/disposed at other location, as noted herein.

As shown in FIG. 13A, during systole, the mitral valve 116 is closed and the aortic valve 118 is open, allowing blood to flow through the device 1100 and the aorta 120, such as through an internal channel within the elastic tube 1102. As blood is flows into the device 1100, the blood/luminal pressure within the elastic tube 1102 increases and the elastic tube 1102 expands, thereby storing blood and energy within the elastic tube 1102 in the form of elastic stretch.

In contrast, as shown in FIG. 13B, during diastole, the mitral valve 116 is open and the aortic valve 118 is closed, allowing blood to flow into the left ventricle 104 from the left atrium 102. Here, the blood/luminal pressure within the elastic tube 1102 decreases, resulting in the elastic tube 1102 radially compressing and releasing the blood into circulation within the aorta 120. That is, the stored energy in the elastic tube 1102 causes elastic tube 1102 to contract/recoil back towards the original tubular state thereof due to decreasing blood pressure within the device 1100.

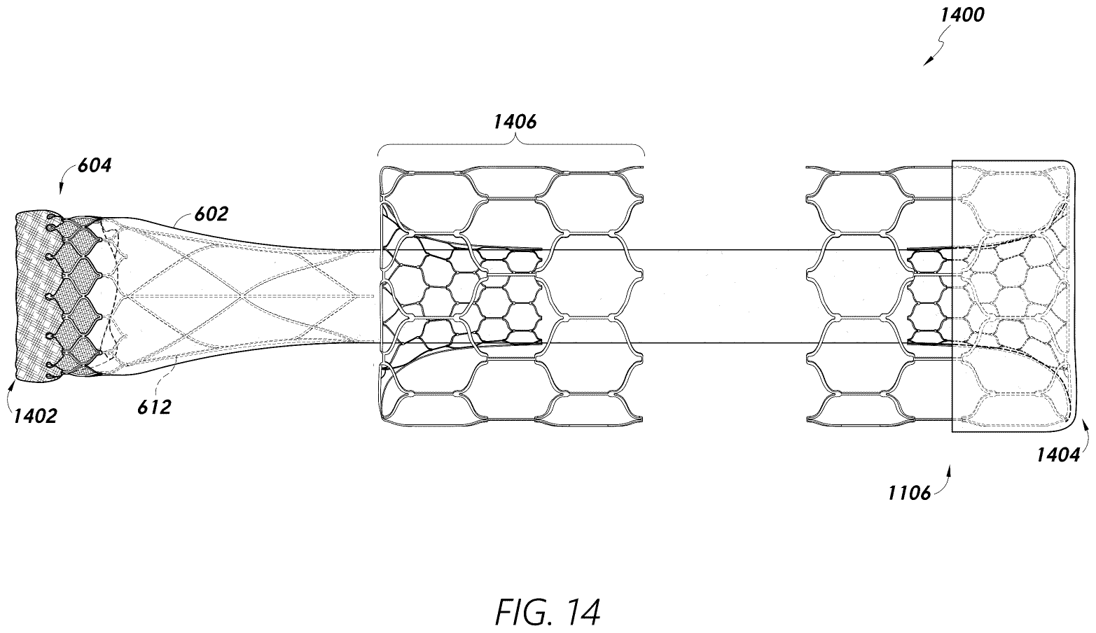

FIG. 14 provides a side view of an example device/system 1400 that can be configured to enhance compliance of a fluid vessel (also referred to as “the implant device 1400”). The device 1400 can include one or more features/elements of the device 600, the device 1100, and/or other features. For ease of discussion, the elements of the device 600/1100 are illustrated; however, the features can be implemented in different manners than that previously discussed/presented.

As shown, the device 1400 can include the prosthetic valve 604 configured to be positioned within the desired anatomy. The device 1400 also includes the elastic tube 602, the elastic tube 1102, the anchoring feature 1104, and/or the anchoring feature 1106. In examples, the elastic tube 602 and the elastic tube 1102 are integrated as a single component/elastic tube that spans from a first end/end portion 1402 of the device 1400 where the prosthetic valve 604 is positioned/disposed to a second end/end portion 1404 of the device 1400 where the anchoring feature 1106 is positioned/disposed. In other examples, the elastic tube 602 and the elastic tube 1102 are implemented as separate components/elastic tubes that include the same or different characteristics. When implemented as separated components, the elastic tubes 602 and 1102 can be coupled together. Whether implemented as integral or separate components, the elastic tubes 602 and 1102 can be attached to the anchoring feature 1104 located in a medial portion 1406 of the device 1400.

The device 1400 can also include the internal frame 612, which can be coupled to or integral with the anchoring feature 1104 and/or the anchoring feature 1106. In examples, the internal frame 612 extends through the entire device 1400, while in other examples the internal frame 612 extends through just a portion. The internal frame 612 can provide support to prevent the elastic tube 602/1102 from collapsing into a fluid channel within the device 1400.

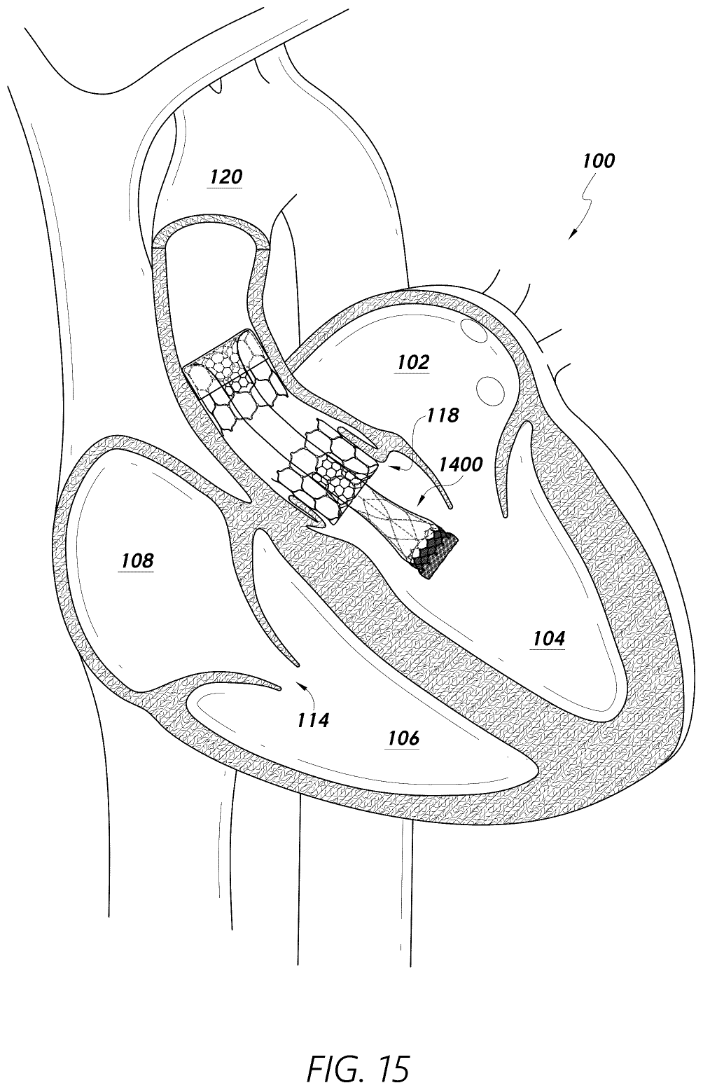

FIG. 15 illustrates the device 1400 disposed/implanted within example anatomy of a patient, namely the left ventricle 104, the aortic valve 118, and the aorta 120 in a cross-sectional view of the heart 100, in accordance with one or more examples. Here, the first end 1402 of the device 1400 is implanted/disposed within the left ventricle 104, the medial portion 1406 is implanted/disposed in the aortic valve 118, and the second end 1404 is implanted/disposed in the aorta 120. The device 1400 can be implemented with or without a second anchoring feature/prosthetic valve (or other feature, such as a valve docking station) at the medical portion 1406 and/or a third anchoring feature/prosthetic valve (or other feature, such as a valve docking station) at the second end 1404. Thus, the device 1400 can be implemented with any number of prosthetic valves, anchoring features, and/or other features. The device 1400 can be implanted to enhance the compliance characteristics of the aorta 120, such as by extending the aorta 120 into the left ventricle 104 with a compliant member (i.e., the compliant tube 602) and/or configuring the aorta 120 to have more compliance.

In some example implementations of FIG. 15, the device 1400 can be implanted in a manner that prevents/minimizes blood flow in an area in the aorta 120 between the elastic tube 1102 and the inner surface of the aorta 120, as similarly discussed above for the device 1100. For example, while implanting the anchoring feature 608 and/or 1404, the blood in an area of the aorta 120 between the elastic tube 1102 and the inner wall of the aorta 120 can be removed and/or the area can be filled with another fluid, as similarly discussed above for the device 1100.

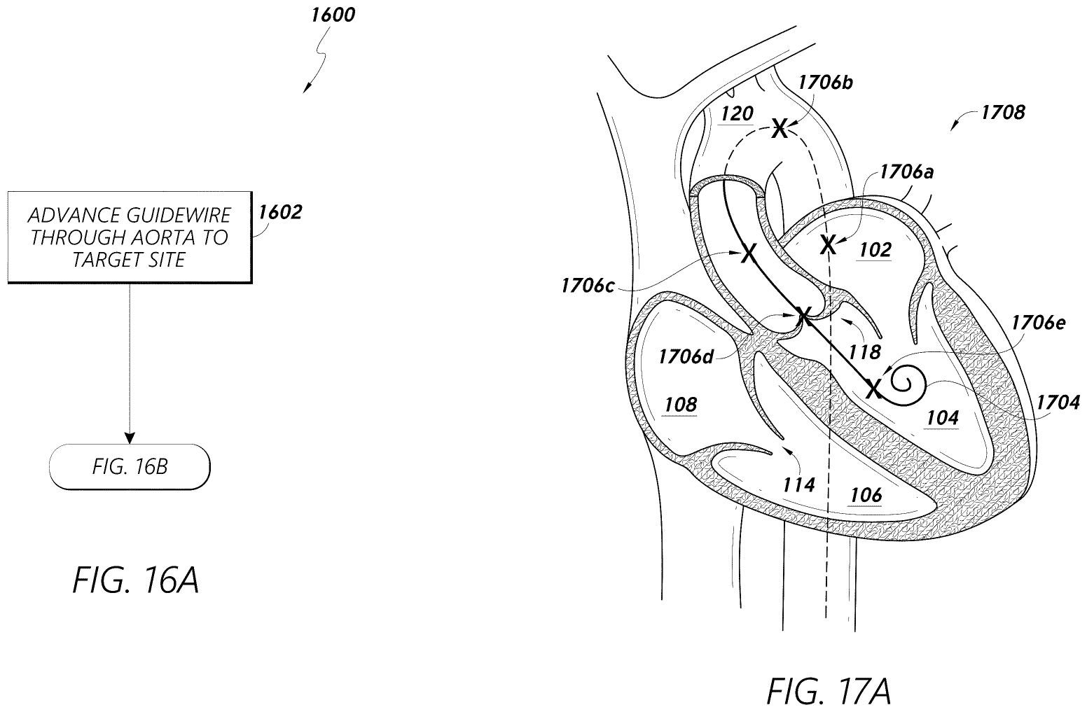

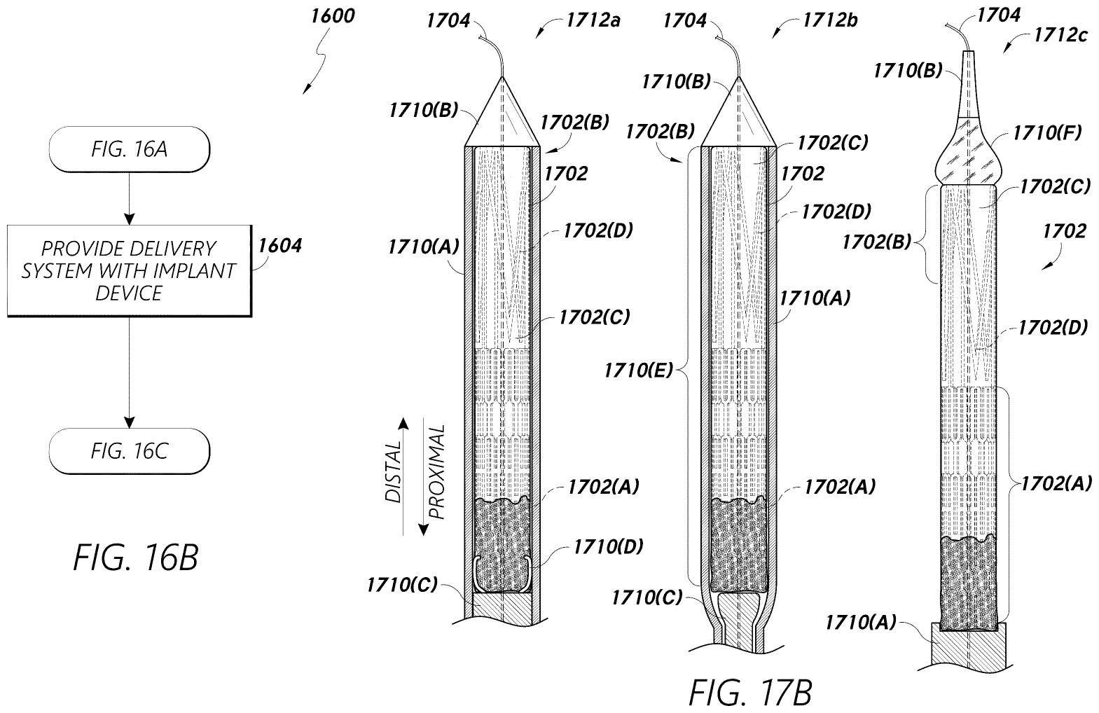

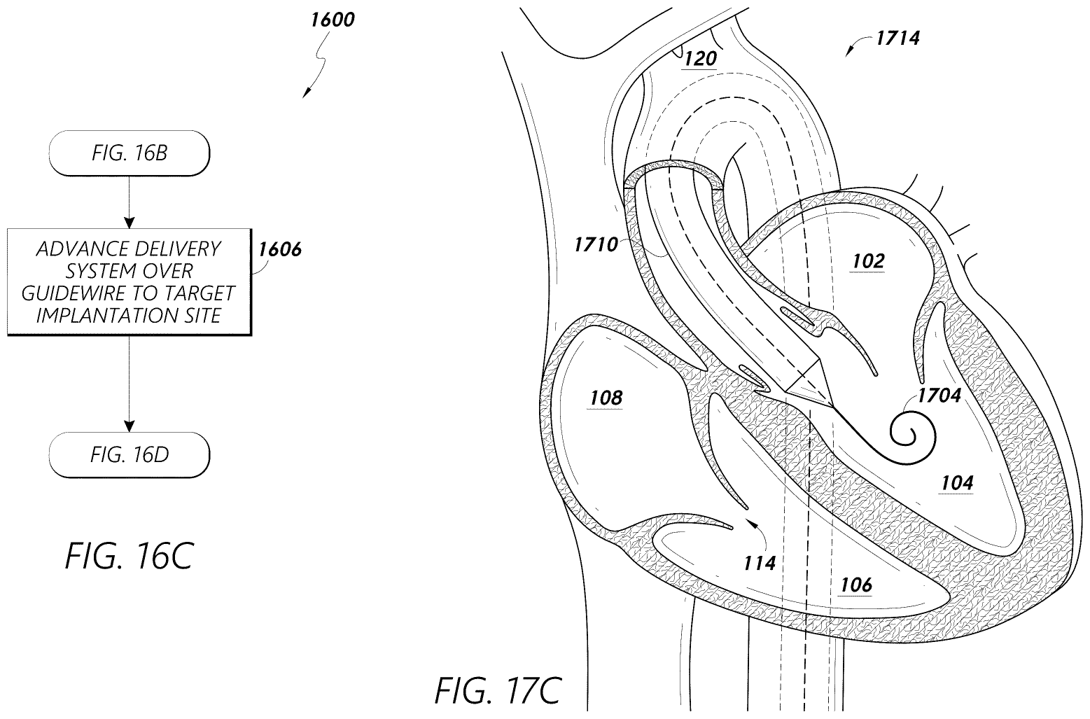

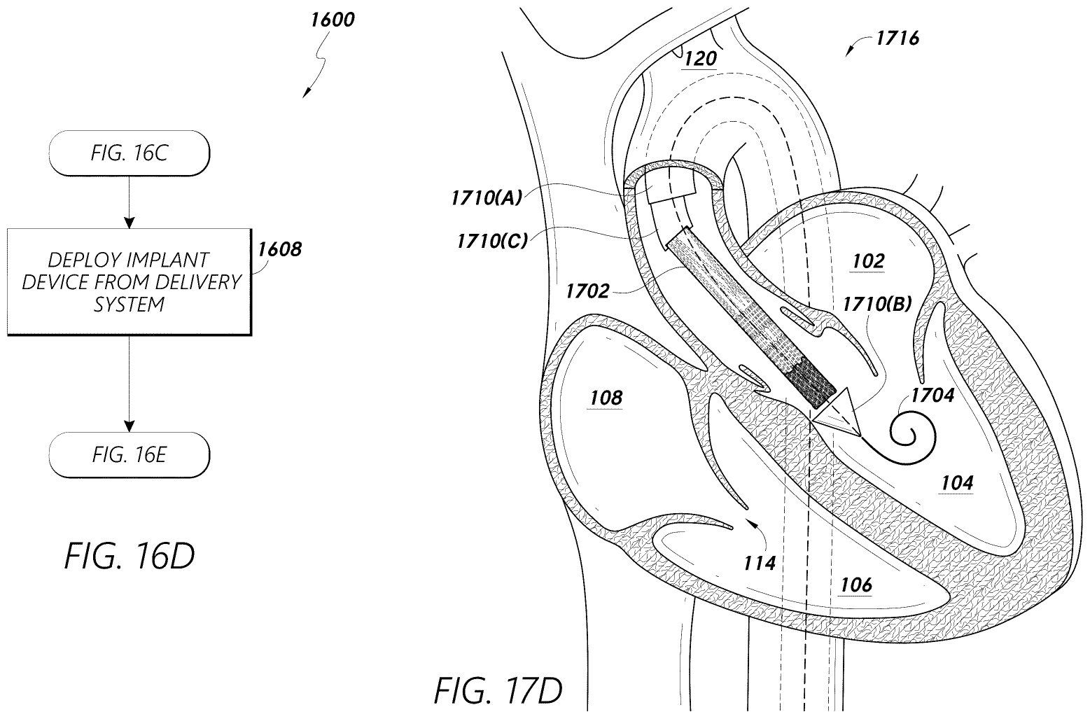

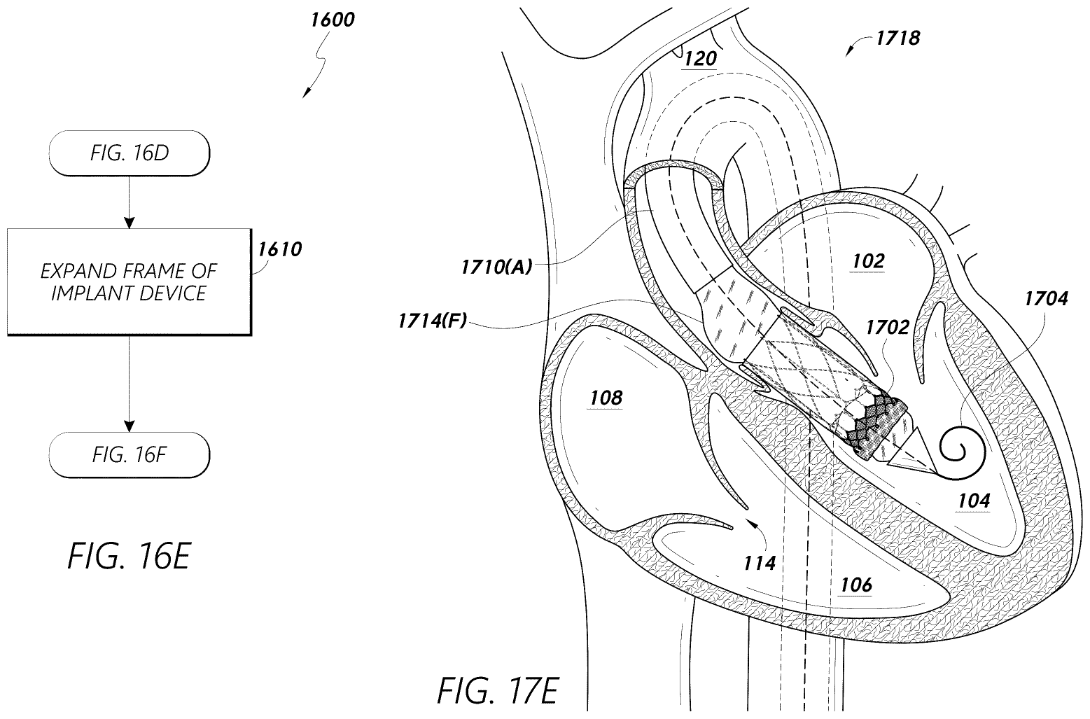

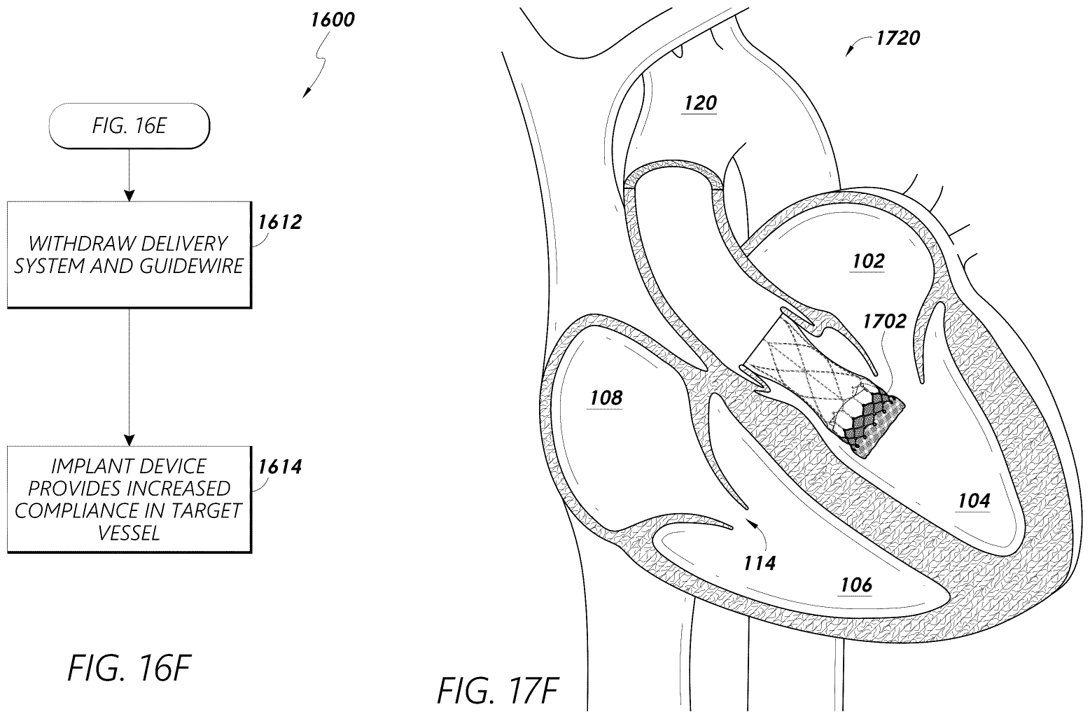

FIGS. 16A, 16B, 16C, 16D, 16E, and 16F illustrate a flow diagram for a process 1600 for implanting an implant device 1702 in accordance with one or more examples. FIGS. 17A, 17B, 17C, 17D, 17E, and 17F provide images of the implant device 1702 and certain anatomy corresponding to operations of the process 1600 of FIGS. 16A, 16B, 16C, 16D, 16E, and 16F according to one or more examples. The process 1600 utilizes a transcatheter procedure for implantation/deployment of compliance-enhancement implant devices in accordance with aspects of the present disclosure. However, implant devices disclosed herein can be implanted using other types of minimally-invasive and/or surgical procedures. The implant device 1702 can be representative of any of the implant devices discussed herein, such as the implant device 600, the implant device 1100, the implant device 1400, or any other implant device.

At block 1602, the process 1600 includes advancing a guidewire 1704 through at least a portion of the aorta 120 and/or the aortic valve 118 of the patient to reach a target implantation site 1706. For example, image 1708 shows an example implantation site 1706a in the descending thoracic aorta, an example implantation site 1706b in the aortic arch, an example implantation site 1706c in the ascending aorta, an example implantation site 1706d in the aortic valve 118, and an example implantation site 1706e in the left ventricle 104. The guidewire 1704 can be advanced through the aortic valve 118 and/or into the left ventricle 104 to the desired implantation site 1706. Access to the implantation site 1706 can be made through any suitable vessel puncture providing access to the arterial system. For example, access can be made via the femoral artery or other arterial blood vessel. In some implementations, access is made to the inferior vena cava via the femoral vein or other access, wherein a guidewire and/or other instrumentation can be crossed over into the abdominal aorta in an area where the inferior vena cava and abdominal aorta are adjacent to one another by puncturing through the venous wall and the arterial wall and advancing through such puncture openings.

Although the process 1600 and certain other examples are described herein in the context of implantation within the aortic valve 118, compliance-enhancement devices of the present disclosure can be implanted in other arterial or venous blood vessels, such as the inferior vena cava. Further, although the process 1600 and accompanying illustrations are presented with respect to the implantation of a single compliance-enhancement implant device 1702, the process 1600 can involve implanting multiple compliance-enhancement implant devices in various positions within the aorta 1706/aortic valve 118 and/or other blood vessel(s)/valve(s).

In FIG. 16B, at block 1604, the process 1600 includes providing a delivery system 1710 having the implant device 1702 disposed in a distal portion thereof. Images 1712a, 1712b, and 1712c of FIG. 17B show a cut-away view of implementations of the delivery system 1710 in accordance with one or more examples. The delivery system 1710 can comprise one or more catheters, sheaths, balloons, and/or other devices used to advance and/or implant the implant device 1702, which can be disposed at least partially within the delivery system 1710 during portions of the process 1600. The implant device 1702 can be positioned within the delivery system 1710 with a first end thereof (e.g., a prosthetic valve 1702(A)) disposed proximally and a second end (e.g., an anchoring feature 1702(B)) disposed distally with respect to the illustrated orientation of the delivery system 1710.

In some examples, the delivery system 1710 comprises an outer catheter/shaft/sheath 1710(A), which can be used to transport the compliance-enhancement implant device 1702 to the target implantation site. That is, the compliance-enhancement implant device 1702 can be advanced to the target implantation site at least partially within a lumen of the outer shaft 1710(A), such that the compliance-enhancement implant device 1702 is held and/or secured at least partially within a distal portion of the outer shaft 1710(A) in a radially compressed configuration.

In some examples, the delivery system 1710 comprises a tapered nosecone feature 1710(B), which can facilitate advancement of the distal end of the delivery system 1710 through the tortuous anatomy of the patient and/or an outer delivery sheath or other conduit/path. The nosecone 1710(B) can be a separate component from the outer shaft 1710(A) or can be integrated with the outer shaft 1710(A). In some examples, the nosecone 1710(B) is adjacent to and/or integrated with a distal end of the outer shaft 1710(A). In some examples, the nosecone 1710(B) is distally tapered into a generally conical shape and can comprise and/or be formed of multiple flap-type forms that can be urged/spread apart when the compliance-enhancement implant device 1702 and/or any portions thereof, interior shafts, or devices, are advanced distally therethrough.

The delivery system 1710 can further be configured to have the guidewire 1704 disposed at least partially within the delivery system 1710 and/or coupled thereto in a manner to allow the delivery system 1710 to follow a path defined by the guidewire 1704. In some implementations, the guidewire 1704 can pass through an interior of the implant device 1702 and/or through a lumen of a pusher device or tube of the delivery system 1710.

The implant device 1702 can have any of the features of the examples described herein, including the prosthetic valve 1702(A) (which can be similar to or the same as the prosthetic valve 604 of the device 600, for example), the anchoring feature 1702(B) (which can be similar to or the same as the anchoring feature 608 of the device 600, for example), an elastic tube 1702(C) (which can be similar to or the same as the elastic tube 602 of the device 600, for example), an internal frame 1702(D) (which can be similar to or the same as the internal frame 612 of the device 600, for example), and/or any other features of the implant devices discussed herein, such as the implant device 1100, the implant device 1400, etc.

In the examples of images 1712a and 1712b, the implant device 1702 is configured to be compressed/collapsed and disposed within the shaft/sheath 1710(A) in the radially compressed/collapsed configuration, wherein the prosthetic valve 1702(A), anchoring feature 1702(B), elastic tube 1702(C), and/or internal frame 1702(D) are crimped to assume a reduce radial profile. In the compressed delivery configuration, the device 1702 can be somewhat more elongated compared to a fully expanded configuration thereof due to at least some of frame structure of the device 1702 being deflected into more longitudinally-oriented configurations. In the example of image 1712c the implant device 1702 can be compressed/collapsed and/or held between a balloon 1710(F) and the sheath 1710(A).

In some cases, the delivery system 1710 can comprise a pusher shaft 1710(C), which can be slidingly disposed within the outer sheath 1710(A) proximal and/or adjacent to the implant device 1702. The pusher 1710(C) can be configured to be used to push/advance the implant device 1702 relative to the outer shaft/sheath 1710(A) to deploy the device 1702 from the sheath 1710(A). For example, the pusher 1710(C) can be distally advanced relative to the outer sheath 1710(A) to cause distal advancement of the implant device 1702 through a distal opening in the outer sheath/shaft 1710(A). Alternatively, or additionally, the implant device 1702 can be deployed from the outer sheath 1710(A) at least in part by proximally pulling the outer sheath 1710(A) relative to the pusher 1710(C).

The image 1712a illustrate an example implementation of the delivery system 1710 that includes one or more coupling/attachment elements 1710(D) to releasably attach to the implant device 1702. For example, the coupling elements 1710(D) can comprise one or more feet or arms that project distally and/or radially from the pusher 1710(C). In some cases, the pusher 1710(C) is releasably attached to the implant device 1702. After the device 1702 has been deployed from the sheath 1710(A), positioned in the desired implantation site/position, and/or expanded, the pusher 1710(C)/coupling element(s) 1710(D) (or other component of the delivery system 1710) can be disengaged from the implant device 1702 to release the device 1702 and allow for removal/withdrawal of the delivery system 1710. Although coupling elements 1710(D) are illustrated, these elements can be eliminated.

The image 1712b shows an example implementation of the delivery system 1710 that includes a distal capsule portion 1710(E), wherein the implant device 1702 is disposed in the compressed configuration within the capsule portion 1710(E). Deployment of the implant device 530 can be implemented at least in part by proximally pulling the outer sheath 1710(A) such that the implant device 1710 is pressed proximally against the pusher 1710(C) to keep the implant device 1710 in place while unsheathing is taking place. In some implementations, the capsule portion 1710(E) can have a diameter that is greater than that of the outer sheath 1710(A) in the area proximal to the capsule 1710(E) (e.g., a more proximal part of the implant device 1710).

The image 1712c shows an example delivery system 1710c that includes the balloon 1710(F), which can be positioned at least partially within the implant device 1702 for delivery of the implant device 1702. Here, the implant device 1702 is configured to remain in a compressed state with minimal or no constraints (e.g., with the outer sheath 1710(A) covering just a portion or none of the implant device 1702), wherein the implant device 1702 is configured to be expanded by the balloon 1710(F) at a target site.

Although features are shown with respect to particular delivery system implementations, any of the delivery systems 1710 of images 1712a-c can include any of the features, even if such feature is not illustrated for a specific implementation.

In FIG. 16C, at block 1606, the process 1600 includes advancing the delivery system 1710 over the guidewire 1704 until the target implantation site is reached to thereby position the implant device 1702 for deployment in the target anatomy, as shown in image 1714 of FIG. 17C. For instance, the delivery system 1710 can be advanced to position the prosthetic valve 1702(A) in the left ventricle 104 and position the anchoring feature 1702(B) at the aortic valve 118; however, other target sites can be used, such as a location within the aorta 120.

In FIG. 16D, at block 1608, the process 1600 includes deploying the implant device 1702 from the delivery system 1710. In examples, to deploy the implant device 1702, the outer sheath 1710(A) is proximally pulled and/or the pusher 1710(C) is distally pushed to thereby draw the sheath 1710(A) past the distal end of the implant device 1702, at least partially exposing/deploying the implant device 1702, as shown in image 1716. For instance, initially the sheath 1710(A) can be withdrawn to position the prosthetic valve 1702(A) within the left ventricle 104 (which can include anchoring the prosthetic valve 1702(A) to the tissue in the left ventricle 104, in some examples), while maintaining position around and holding the anchoring feature 1702(B). The sheath 1710(A) can be further withdrawn to position/anchor the anchoring feature 1702(B) at the aortic valve 118. In some cases, such as when the example implant device 1400 is deployed, the sheath 1710(A) can be further withdrawn to position an additional anchoring feature for the implant device 1702 within the aorta 120. The implant device 1702 can comprise one or more radiopaque markers that can be referenced to determine/confirm the position of the implant device 1702 at various stage(s) of the process 1600 using a suitable imaging modality.

Although discussed in the context of implanting a device within the left ventricle 104 and aortic valve 118, the implant device 1702 can be implanted in other anatomy. For instance, the implant device 1702 can be representative of the implant device 1100, wherein the implant device 1100 can be positioned within the delivery system 1710 with the anchoring feature 1104 positioned distally and the anchoring feature 1106 positioned proximally. Here, the implant device 1100 can be implanted within the aorta 120 by first implanting the anchoring feature 1104 in the aortic valve 118 or the aorta 120, which can include proximally pulling the outer sheath 1710(A) and/or distally pushing the pusher 1710(C) to draw the sheath 1710(A) past the anchoring feature 1104. The anchoring feature 1106 can then be positioned within the aorta 120 in a similar fashion by further proximally pulling the outer sheath 1710(A) and/or distally pushing the pusher 1710(C).

The prosthetic valve 1702(A), anchoring feature 1702(B), elastic tube 1702(C), internal frame 1702(D), and/or other elements of the implant device 1702 can be implemented in a variety of manners. In examples, any of these elements can be self-expandable, such that portion of the implant device 1702 is expanded/fully deployed (e.g., anchored/secured to target tissue) upon release from the outer sheath 1710(A). For instance, expansion of a portion of the implant device 1702 can be achieved via shape memory features. To illustrate, one or more portions of the device 1702 can comprise nitinol or other shape-memory metal configured to self-expand when released from the delivery sheath/capsule.

Further, in examples, the prosthetic valve 1702(A), anchoring feature 1702(B), elastic tube 1702(C), internal frame 1702(D), and/or other elements of the implant device 1702 can be expanded by a device/physician. In such cases, in FIG. 16E, at block 1610, the process 1600 includes expanding an element(s) of the implant device 1702 to thereby secure the implant device 1702 in place in the deployed/expanded configuration.

In examples, as shown in image 1718, the delivery system 1710 includes the balloon 1710(F) configured to expand one or more elements/portions of the implant device 1702. The implant device 1702 can be situated on the balloon 1710(F) such that when the balloon 1710(F) is inflated, one or more portions of the implant device 1702 expand with the balloon 1710(F). The balloon 1710(F) can serve to expand frame struts/portions by pushing outwardly against the frame struts/portions. In examples, the balloon 1710(F) is positioned within the internal frame 1702(D), when such internal frame is implemented. After the balloon 1710(F) has expanded the implant device 1702, the balloon 1710(F) can be deflated and removed.

Further, in examples, the implant device 1702 or any elements/portions thereof can be expanded/dilated using pull wire(s) that are configured to be pulled or pushed to cause expansion of element(s). For example, a pull wire(s) can be coupled to the distal portion of an element(s) of the implant device 1702 such that pulling the wire(s) proximally causes the ends of the element(s) to be brought together, thereby dilating/expanding the element(s).

In some cases, the expansion operation(s) associated with block 1610 can involve dilating the native valve/vessel (before or after expanding the implant device 1702) by expanding the implant device 1702 or portion thereof to a diameter, at least with respect to a lengthwise portion thereof, that is greater than the diameter of the native valve/vessel, wherein such dilation of the valve/vessel can serve to form/introduce a space for radial expansion of the elastic tube 1702(C) and/or one or more portions of the implant device 1702.

In FIG. 16F, at block 1612, the process 1600 includes withdrawing the delivery system 1710 and/or guidewire 1704, leaving the implant device 1702 implanted, as shown in image 1720. With the implant device 1702 implanted, the implant device 1702 can provide increased compliance to thereby improve arterial blood flow and/or prevent elevated blood pressure, as shown at block 1614. Other benefits can also be achieved, as described herein.

Any of the various systems, devices, apparatuses, etc. in this disclosure can be sterilized (e.g., with heat, radiation, ethylene oxide, hydrogen peroxide, etc.) to ensure they are safe for use with patients, and/or the methods herein can comprise sterilization of the associated system, device, apparatus, etc. (e.g., with heat, radiation, ethylene oxide, hydrogen peroxide, etc.).

### Additional Description of Examples

Provided below is a list of examples, each of which may include aspects of any of the other examples disclosed herein. Furthermore, aspects of any example described above may be implemented in any of the numbered examples provided below.

### Example 1

An implant device comprising: a first elastic tube including a first end portion and a second end portion; a first anchoring structure associated with the first end portion and configured to anchor the implant device; and a first prosthetic valve coupled to the second end portion.

### Example 2

The implant device of any example herein, in particular example 1, wherein the implant device is configured to extend from the first anchoring structure into a heart chamber.

### Example 3

The implant device of any example herein, in particular example 1 or example 2, further comprising: a frame including a cylindrical form, the frame being disposed within the first elastic tube and configured to prevent the first elastic tube from radially compressing.

### Example 4

The implant device of any example herein, in particular example 3, further comprising: a second elastic tube including a first end portion and a second end portion, the first end portion of the second elastic tube being coupled to the first end portion of the first elastic tube.

### Example 5

The implant device of any example herein, in particular example 4, further comprising: a second anchoring structure associated with the second end portion of the second elastic tube and configured to anchor the second elastic tube to a blood vessel.

### Example 6

The implant device of any example herein, in particular examples 1-5, further comprising: a second prosthetic valve coupled to the first end portion of the first elastic tube.

### Example 7

The implant device of any example herein, in particular example 6, wherein the first prosthetic valve is configured with a first blood flow characteristic that is based at least in part on a second blood flow characteristic of the second prosthetic valve.

### Example 8

The implant device of any example herein, in particular examples 1-7, wherein the first prosthetic valve includes a second anchoring structure configured to anchor the first prosthetic valve to tissue within a heart chamber.

### Example 9

The implant device of any example herein, in particular examples 1-8, further comprising: a valve docking station coupled to the first anchoring structure and configured to receive a second prosthetic valve.

### Example 10

The implant device of any example herein, in particular examples 1-9, wherein the first anchoring structure is configured to anchor the implant device within an aortic valve.

### Example 11

The implant device of any example herein, in particular examples 1-10, wherein the implant device is sterilized.

### Example 12

A device comprising: a first elastic conduit configured to anchor to a heart valve area and extend into a heart chamber; and a first prosthetic valve coupled to a portion of the first elastic conduit that is configured to be positioned within the heart chamber.

### Example 13

The device of any example herein, in particular example 12, further comprising: a frame disposed within the first elastic conduit and configured to prevent the first elastic conduit from radially compressing.

### Example 14

The device of any example herein, in particular examples 12 or 13, further comprising: an anchor coupled to the first elastic conduit and configured to anchor the device to the heart valve area.

### Example 15

The device of any example herein, in particular examples 12-14, further comprising: a second elastic conduit coupled to the first elastic conduit and configured to extend into a blood vessel associated with the heart chamber.

### Example 16

The device of any example herein, in particular example 15, further comprising: an anchor coupled to the second elastic conduit and configured to anchor the second elastic conduit to the blood vessel.

### Example 17

The device of any example herein, in particular examples 12-16, wherein the first prosthetic valve is coupled to a first end of the first elastic conduit, and the device further comprises: a second prosthetic valve coupled to a second end of the first elastic conduit.

### Example 18

The device of any example herein, in particular examples 12-17, wherein the first prosthetic valve is configured with a first blood flow characteristic that is based at least in part on a second blood flow characteristic of the second prosthetic valve.

### Example 19

The device of any example herein, in particular examples 12-18, wherein the first elastic conduit is configured to anchor to the heart chamber.

### Example 20

The device of any example herein, in particular examples 12-19, wherein the first prosthetic valve is coupled to a first end of the first elastic conduit, and the device further comprises: a valve docking station coupled to a second end of the first elastic conduit and configured to receive a second prosthetic valve.

### Example 21

The device of any example herein, in particular examples 12-20, wherein the heart valve area is associated with an aortic valve.

### Example 22

The device of any example herein, in particular examples 12-21, wherein the device is sterilized.

### Example 23

A method comprising: advancing an implant device through a fluid vessel, the implant device including a first elastic conduit and a first prosthetic valve coupled to a first end of the first elastic conduit; and deploying the implant device by anchoring at least a portion of the first elastic conduit within a heart valve.

### Example 24

The method of any example herein, in particular example 23, wherein the deploying includes positioning the first end of the first elastic conduit within a heart chamber.

### Example 25

The method of any example herein, in particular example 23 or example 24, wherein the deploying includes anchoring a second end of the first elastic conduit to the heart valve.

### Example 26

The method of any example herein, in particular examples 23-25, wherein the implant device includes a frame disposed within the first elastic conduit and configured to prevent the first elastic conduit from radially compressing.

### Example 27

The method of any example herein, in particular examples 23-26, wherein the implant device includes an anchor coupled to the first elastic conduit and configured to anchor the implant device to the heart valve.

### Example 28

The method of any example herein, in particular examples 23-27, wherein the implant device includes a second elastic conduit coupled to the first elastic conduit and configured to extend into a blood vessel associated with the heart valve.

### Example 29

The method of any example herein, in particular example 28, wherein the implant device includes an anchor coupled to the second elastic conduit and configured to anchor the second elastic conduit to the blood vessel.

### Example 30

The method of any example herein, in particular examples 23-29, wherein the implant device includes a second prosthetic valve coupled to a second end of the first elastic conduit.

### Example 31

The method of any example herein, in particular example 30, wherein the first prosthetic valve is configured with a first blood flow characteristic that is based at least in part on a second blood flow characteristic of the second prosthetic valve.

### Example 32

The method of any example herein, in particular examples 23-31, wherein the first end of the first elastic conduit is configured to anchor to a heart chamber.

### Example 33

The method of any example herein, in particular examples 23-32, wherein the implant device further includes a valve docking station coupled to a second end of the first elastic conduit and configured to receive a second prosthetic valve.

### Example 34

The method of any example herein, in particular examples 23-33, wherein the heart valve is an aortic valve.

### Example 35

The method of any example herein, in particular examples 23-34, wherein the implant device is sterilized.

### Example 36

An implant device comprising: a first anchoring structure; a second anchoring structure; and an elastic tube coupled between the first anchoring structure and the second anchoring structure to provide a channel through the first anchoring structure and the second anchoring structure.

### Example 37

The implant device of any example herein, in particular example 36, wherein the first anchoring structure includes an inner frame structure that has a smaller diameter than an outer diameter of the first anchoring structure, the elastic tube being coupled to the inner frame structure.

### Example 38

The implant device of any example herein, in particular example 36 or example 37, further comprising: a first prosthetic valve associated with the first anchoring structure.

### Example 39

The implant device of any example herein, in particular example 38, further comprising: a valve docking station associated with the second anchoring structure and configured to receive a second prosthetic valve.

### Example 40

The implant device of any example herein, in particular example 38, further comprising: a second prosthetic valve associated with the second anchoring structure.

### Example 41

The implant device of any example herein, in particular example 40, wherein the first prosthetic valve is configured with a first blood flow characteristic that is based at least in part on a second blood flow characteristic of the second prosthetic valve.

### Example 42

The implant device of any example herein, in particular examples 36-41, further comprising: a valve docking station associated with the first anchoring structure and configured to receive a prosthetic valve.

### Example 43

The implant device of any example herein, in particular examples 36-42, wherein at least one of the first anchoring structure or the second anchoring structure includes an expandable frame.

### Example 44

The implant device of any example herein, in particular examples 36-43, wherein at least one of the first anchoring structure or the second anchoring structure includes shape-memory metal.

### Example 45

A method comprising: advancing an implant device through a fluid vessel, the implant device including a first anchoring structure, a second anchoring structure, and an elastic tube coupled between the first anchoring structure and the second anchoring structure to provide a channel through the first anchoring structure and the second anchoring structure; and deploying the implant device by anchoring at least one of the first anchoring structure or the second anchoring structure within the fluid vessel.

### Example 46

The method of any example herein, in particular example 45, wherein the first anchoring structure includes an inner frame structure that has a smaller diameter than an outer diameter of the first anchoring structure, the elastic tube being coupled to the inner frame structure.

### Example 47

The method of any example herein, in particular example 45 or example 46, wherein the implant device further includes a first prosthetic valve associated with the first anchoring structure.

### Example 48

The method of any example herein, in particular example 47, wherein the implant device further includes a valve docking station associated with the second anchoring structure and configured to receive a second prosthetic valve.

### Example 49

The method of any example herein, in particular example 47, wherein the implant device further includes a second prosthetic valve associated with the second anchoring structure.

### Example 50

The method of any example herein, in particular example 49, wherein the first prosthetic valve is configured with a first blood flow characteristic that is based at least in part on a second blood flow characteristic of the second prosthetic valve.

### Example 51

The method of any example herein, in particular examples 45-50, wherein the implant device further includes a valve docking station associated with the first anchoring structure and configured to receive a prosthetic valve.

### Example 52

The method of any example herein, in particular examples 45-51, wherein at least one of the first anchoring structure or the second anchoring structure includes an expandable frame.

### Example 53

The method of any example herein, in particular examples 45-52, wherein at least one of the first anchoring structure or the second anchoring structure includes shape-memory metal.

Depending on the example, certain acts, events, or functions of any of the processes or algorithms described herein can be performed in a different sequence, can be added, merged, and/or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, can encompass the concepts of equality. For example, “less” can mean not only “less” in the strictest mathematical sense, but also “less than or equal to.”

What is claimed is:

1. An implant device comprising:
a first elastic tube including a first end portion and a second end portion, the first elastic tube adapted to extend from the first end portion into a heart chamber;
a first anchoring structure associated with the first end portion and dimensioned to be anchored to a native heart valve associated with the heart chamber; and
a first prosthetic valve coupled to the second end portion, the first prosthetic valve adapted to be disposed within the heart chamber.

2. The implant device of claim 1, further comprising:
a frame including a cylindrical form, the frame being disposed within the first elastic tube and dimensioned to prevent the first elastic tube from radially compressing.

3. The implant device of claim 2, further comprising:
a second elastic tube including a first end portion and a second end portion, the first end portion of the second elastic tube being coupled to the first end portion of the first elastic tube.

4. The implant device of claim 3, further comprising:
a second anchoring structure associated with the second end portion of the second elastic tube and dimensioned to anchor the second elastic tube to a blood vessel.

5. The implant device of claim 1, further comprising:
a second prosthetic valve coupled to the first end portion of the first elastic tube.

6. The implant device of claim 5, wherein the first prosthetic valve includes a first blood flow characteristic that is based at least in part on a second blood flow characteristic of the second prosthetic valve.

7. The implant device of claim 1, wherein the first prosthetic valve includes a second anchoring structure configured to anchor the first prosthetic valve to tissue within the heart chamber.

8. The implant device of claim 1, further comprising:
a valve docking station coupled to the first anchoring structure and adapted to receive a second prosthetic valve.

9. An implant device comprising:
a first anchoring feature adapted to be anchored to a heart valve;
a first compliant tube coupled to the first anchoring feature at a first end portion of the first compliant tube; and
a prosthetic valve coupled to a second end portion of the first compliant tube, the prosthetic valve adapted to be disposed within a heart chamber.

10. The implant device of claim 9, further comprising:
a frame disposed within the first compliant tube and dimensioned to maintain a minimum diameter for the first compliant tube when the first compliant tube radially compresses.

11. The implant device of claim 9, further comprising:
a second compliant tube including a first end portion and a second end portion, the first end portion of the second compliant tube being coupled to the prosthetic valve; and
a second anchoring feature associated with the second end portion of the second compliant tube and dimensioned to anchor the second compliant tube to a blood vessel.

12. The implant device of claim 9, wherein the prosthetic valve includes a second anchoring feature configured to anchor the prosthetic valve to tissue within the heart chamber.

13. The implant device of claim 9, further comprising:
a valve docking station coupled to the first anchoring feature and adapted to receive another prosthetic valve.

14. The implant device of claim 9, wherein the heart valve is an aortic valve.

15. An implant device comprising:
a first anchoring structure adapted to anchor to a heart chamber;
a first prosthetic valve coupled to the first anchoring structure;
a second anchoring structure adapted to anchor to a native heart valve; and
an elastic tube coupled between the first anchoring structure and the second anchoring structure to provide a channel to an output blood vessel associated with the native heart valve.

16. The implant device of claim 15, wherein the first anchoring structure includes an inner frame structure that has a smaller diameter than an outer diameter of the first anchoring structure, the elastic tube being coupled to the inner frame structure.

17. The implant device of claim 15, further comprising:
a valve docking station associated with the second anchoring structure and adapted to receive a second prosthetic valve.

18. The implant device of claim 15, further comprising:
a second prosthetic valve associated with the second anchoring structure.

19. The implant device of claim 18, wherein the first prosthetic valve includes a first blood flow characteristic that is based at least in part on a second blood flow characteristic of the second prosthetic valve.

20. The implant device of claim 15, wherein at least one of the first anchoring structure or the second anchoring structure includes an expandable frame.
